(19) United States
(12) Patent Application Publication
(10) Pub. No.: US 2026/0265815 A1
(43) Pub. Date: Sep. 10, 2026

(54) INTEGRATED, MULTI-INSTRUMENT SYSTEM FOR AUTOMATED BIOPROCESSING

(71) Applicant: Cellares Corporation, South San Francisco, CA (US)

(72) Inventors: Daniele MALLEO, Menlo Park, CA (US); Fabian G. GERLINGHAUS, San Francisco, CA (US)

(21) Appl. No.: 19/278,672

(22) Filed: Jul. 23, 2025

Related U.S. Application Data
(60) Provisional application No. 63/768,134, filed on Mar. 6, 2025.

(51) Int. Cl. C12Q 1/6848; B01L 1/04; B01L 3/00

(52) U.S. Cl. CPC C12Q 1/6848; B01L 1/04; B01L 3/5085; B01L 2200/028

(57) ABSTRACT

Systems, devices, and methods for automated bioprocessing are provided. A system for automated bioprocessing includes a pre-processing instrument, a post-processing instrument, and a plurality of processing instruments housed within a support unit. Each processing instrument integrates a plurality of nucleic acid processing steps—from template amplification to lipid nanoparticle encapsulation—into a single, closed-loop operation. The system utilizes self-contained cartridges that interface with processing instruments to execute complete manufacturing sequences without manual intervention. This architecture enables parallel production of personalized nucleic acid therapeutics at commercially viable scales while dramatically reducing facility footprint, contamination risk, and labor requirements compared to traditional manufacturing approaches.

### CROSS REFERENCE TO RELATED APPLICATIONS

This application claims priority to U.S. Provisional Patent Application No. 63/768,134 filed Mar. 6, 2025, the content of which is incorporated herein by reference in its entirety for all purposes.

### TECHNICAL FIELD

Systems, devices, and methods herein relate to automated bioprocessing systems for manufacturing therapeutics, and particularly small-volume therapeutics.

### BACKGROUND

In recent years, much interest has developed in using nucleic acid materials for treating and preventing a variety of diseases. Traditional manufacturing of these therapeutics occurs in large-scale facilities designed to produce thousands or millions of standardized doses, such as for broad public health campaigns. Messenger RNA (mRNA) products hold significant promise in addressing a wide range of medical needs. mRNA manufacturing typically involves the following steps: (1) amplifying a DNA template for a target mRNA, (2) transcribing the mRNA using the DNA template, (3) purifying the mRNA to remove unreacted reagents and impurities, (4) encapsulating the mRNA in carriers such as lipid nanoparticles (LNPs) to form a particle capable of entering cells, and (5) performing sterile filtration to ensure sterility of the final product. While large-scale manufacturing approaches may be able to process one version of this lengthy workflow, current solutions are poorly suited to the emerging field of personalized medicine, where each patient may require a uniquely formulated therapeutic, and where each therapeutic may require a tailored processing workflow.

Current approaches to personalized mRNA manufacturing typically rely on adapting large-scale production equipment designed for batch processing. These approaches suffer from inefficient scale-down, increased cross-contamination risk, and complex logistical challenges when handling multiple patient-specific starting materials. Existing systems also frequently require manual transfers between multiple processing steps, creating opportunities for contamination and human error. Traditional manufacturing facilities use fixed-capacity designs that struggle to accommodate fluctuating demand for personalized therapies, resulting in underutilization during low-demand periods and capacity constraints during high-demand periods.

Producing patient-specific formulations necessitates a manufacturing process that is not only automated and sterile but also highly flexible and capable of producing small, customized batches without cross-contamination while maintaining consistent quality across diverse products. These requirements introduce numerous challenges for current systems, including higher risk of cross-contamination, greater process variability, and increased operator error when conventional large-scale equipment is scaled down. These factors, along with the extensive manual handling requirements and linear process flows of conventional facilities, render personalized nucleic acid therapeutic production not only impractical, but unfeasible to attempt within traditional manufacturing frameworks.

The time-sensitive nature of personalized therapeutics adds another layer of complexity, as rapid turnaround times are often critical to clinical efficacy. Current mRNA processing solutions are largely precluded from efficiently producing small batches due to the scale of system components, high labor requirements, and increased contamination risks. The physical size of components used in traditional systems is simply incompatible with small-volume manufacturing, resulting in limited throughput and complex quality control protocols for small batches.

Accordingly, there is a need for an integrated manufacturing system for producing small-volume, customized nucleic acid therapeutics (e.g., mRNA products) in a timely, reliable, and scalable manner while upholding industry standards for sterility, quality control, and throughput.

### SUMMARY

A system or platform for manufacturing nucleic acid therapeutics is described herein. The system may comprise three classes of instruments: a pre-processing instrument configured to load or seal reagents into a cartridge, a processing instrument configured to execute nucleic acid manufacturing steps on the cartridge, and a finishing instrument configured to extract product from the processed cartridge, perform quality control, and/or package the product. Together, these three instruments may automate an entire nucleic acid bioprocessing workflow, including preparing the cartridge, manufacturing the nucleic acid product, and extracting and finishing the product.

An illustrative system comprising one or more processing instruments for manufacturing e.g., nucleic acid material, may include a plurality of processing instruments arranged on a support unit, where each processing instrument may have a receiving bay for a cartridge and one or more actuating modules configured to execute a bioprocessing workflow. The system may be designed so that each processing instrument may receive its own cartridge and carry out processing steps without requiring the cartridge to be moved once engaged. In some variations, the system may be housed within an enclosure configured to maintain a sterile environment, which may include one or more distinct rooms. Each processing instrument may include a sensor system for monitoring processing in real time, where the sensor system may incorporate various sensor types such as temperature sensors, cameras, or conductivity sensors. In some variations, the processing instrument may include a plurality of thermal elements on opposite sides of the receiving bay to achieve localized temperature control. The receiving bay may provide a combination of mechanical, electrical, or fluid connections to the cartridge. In certain implementations, the processing instrument may include a vent in its sidewall or a door coupled to the receiving bay opening to ensure controlled access to the cartridge. The support unit itself may be formed as a frame or rack, and in some variations, it may comprise stacked slots arranged vertically, where each slot may be sized to hold a single processing instrument. A plurality of support units may be combined or clustered together to expand the system's throughput, such as aligning them in rows or arrays. The system may also include a robot for transferring cartridges to and from the processing instruments and a controller configured to communicate with the actuating modules, where the controller may generate an electronic batch record for the RNA product of each cartridge. The nucleic acid material processed by the system may include material for mRNA production.

A self-contained cartridge for bioprocessing may include a plurality of modules enclosed within a housing configured to maintain a closed environment. The modules may enable operations such as amplification, transcription, digestion, capping and tailing, and purification, with fluid flow managed by channels, chambers, and valves. In some variations, a top portion and bottom portion of the housing may be sealed together to preserve sterility and minimize contamination. The cartridge may also include mechanical, electrical, or fluid interfaces that align with corresponding connectors on the processing instrument, as well as temperature-sensitive elements or waste reservoirs. In certain implementations, the housing may have optically transparent regions for process monitoring, or it may contain one or more internal sensors for measuring parameters such as pH, pressure, flow rate, or temperature. The cartridge may be disposable after a single processing run and may contain template DNA for mRNA synthesis. In some variations, the modules may be arranged to allow sequential steps without transferring the material to another device, and the cartridge may include additional features such as mixing elements or membranes for filtration. The cartridge may be fully self-contained, carrying all necessary reagents and configured to accept thermal inputs through heat-conductive portions of its housing.

Methods for processing nucleic acid material in parallel may include loading a first cartridge with a first nucleic acid material into a first processing instrument and a second cartridge with a second nucleic acid material into a second processing instrument. By executing workflows in parallel, the system may produce separate therapeutic products within a timeframe of about 5 to about 15 hours. In some variations, the nucleic acid materials may target different sequences but follow similar processing steps, which may include in vitro transcription, purification, and lipid nanoparticle encapsulation. The cartridges may remain engaged with their respective processing instruments throughout the workflow, and sensors may provide real-time feedback on process parameters. In certain implementations, the resulting products may be patient-specific formulations. Additional methods may include transferring reagents to each cartridge prior to engagement with the processing instruments and maintaining a closed environment inside each cartridge to prevent contamination. The workflows may consist of a plurality of steps performed sequentially under the control of the system's actuating modules and sensor feedback.

### BRIEF DESCRIPTION OF THE DRAWINGS

FIG. 1A is a block diagram of an exemplary variation of an automated bioprocessing system. FIG. 1B is a block diagram of an exemplary variation of a cartridge of the automated bioprocessing system. FIG. 1C is a block diagram of an exemplary variation of a preparation instrument of the automated bioprocessing system. FIG. 1D is a block diagram of an exemplary variation of a processing instrument of the automated bioprocessing system. FIG. 1E is a block diagram of an exemplary variation of a finishing instrument of the automated bioprocessing system. FIG. 1F is a block diagram of an exemplary variation of a control system of the automated bioprocessing system.

FIG. 2 depicts a perspective view of an exemplary variation of an automated bioprocessing system that comprises a robotic transport system.

FIG. 3 depicts an exploded view of a rendering of an exemplary variation of a cartridge.

FIG. 4 depicts an exploded view of a rendering of an exemplary variation of a processing instrument.

FIG. 5 depicts a perspective view of an illustration of an exemplary variation of a processing instrument comprising a cartridge therein.

FIG. 6 depicts an exemplary variation of a processing workflow executed by processing instruments.

FIG. 7 depicts an exemplary variation of an internal configuration of a preparation instrument.

FIG. 8 depicts an exemplary variation of a processing workflow executed by a preparation instrument.

FIG. 9 depicts an exemplary variation of an internal configuration of a finishing instrument.

FIG. 10A depicts an exemplary variation of a processing workflow executed by a finishing instrument. FIG. 10B depicts a top view of the finishing instrument used to execute the workflow of FIG. 10A.

FIG. 11 is a schematic diagram of an exemplary variation of a configuration of support units within an automated bioprocessing system.

FIG. 12A is a flow diagram of an exemplary variation of a method for automated bioprocessing. FIG. 12B is another flow diagram of the method for automated bioprocessing.

FIG. 13 is a schematic diagram of an exemplary variation of a data flow path during automated bioprocessing.

### DETAILED DESCRIPTION

Disclosed herein are systems, devices, and methods for automated, integrated small-volume manufacturing of nucleic acid therapeutics. In particular, this disclosure addresses the limitations of conventional large-scale manufacturing with an automated, integrated system that supports high-throughput small-volume production of therapeutics. The system may be configured to carry out all processing steps for nucleic acid therapeutics (e.g., mRNA) in a single, self-contained workflow, thereby minimizing manual handling and reducing the risk of contamination. The modular system architecture described herein may enable parallel processing of a plurality of patient-specific, “lot of one” batches while maintaining system-wide, scalable integration. In some variations, therapeutic products provided via the system herein may be produced within a timeframe of about 5 hours to about 15 hours. Accordingly, the high-throughput system may be configured to meet time and scalability demands for a plurality of custom therapeutics.

The system or platform may comprise three classes of instruments: a pre-processing instrument (“preparation instrument”) configured to load or seal reagents into a cartridge, a processing instrument (“manufacturing instrument”) configured to execute nucleic acid manufacturing steps on the cartridge, and a finishing instrument (“post-processing instrument”) configured to extract product from the processed cartridge, perform quality control, and/or package the product. Together, these three instruments may automate an entire nucleic acid bioprocessing workflow, including preparing the cartridge (pre-processing), manufacturing the nucleic acid product (processing), and extracting and finalizing the product (post-processing). A single cartridge may therefore advantageously transition through multiple states (blank to filled/prepared to processed to finished), correlating to each instrument's role. By maintaining a closed workflow across these steps, the system may reduce contamination risk, simplify batch tracking, and facilitate parallel production of numerous patient-specific batches under a unified control framework. Advantageously, in some variations, the system is a closed system that enables all of the pre-processing, processing, and post-processing stages to be performed in an automated fashion. In other variations, the system enables each of the pre-processing, processing, and post-processing stages to be performed automatically.

The processing instrument may be sized and shaped to achieve small-volume product manufacturing. The system may comprise a configurable, compact design that accommodates a plurality of such processing instruments in a stacked configuration. This arrangement may reduce a total footprint of the instrumentation within the system. In particular, the system may further comprise support units configured to support clusters comprising a plurality of processing instruments. For example, a support unit, which may comprise a rigid frame or rack, may comprise a plurality of slots, each configured to house a processing instrument therein. This structural layout may optimize the use of manufacturing space and create a practical, datacenter-like environment where numerous small-volume manufacturing runs may be executed in parallel. Additionally, this layout may significantly reduce construction and operating costs compared to expanding large-scale production lines.

Each processing instrument may be configured to engage with a filled, pre-processed cartridge that includes most or all necessary reagents and materials to produce the nucleic acid product from start to finish. This may enable a single processing instrument to execute an entire workflow via a cartridge without the need for moving the cartridge among specialized instruments or transferring reagents/wastes in and out of the cartridge. By keeping the cartridge in a closed, self-contained state and minimizing these transfers, the system may advantageously reduce contamination risks, simplify batch tracking, and achieve faster turnaround times. This design may be conducive to parallel processing, enabling a plurality of small-batch workflows to proceed simultaneously.

A single cartridge may transition through multiple states throughout the workflow. Initially, the cartridge may be in a “blank” state, meaning it lacks reagents and may be unsealed. The blank cartridge may be transferred into the system enclosure and then to a preparation instrument, which may load and seal the cartridge with the necessary materials and optionally prime or thermally condition them, resulting in a “filled” or “prepared” cartridge. The filled cartridge may then be transferred to a processing instrument, which may perform nucleic acid manufacturing steps (e.g., amplification, transcription, purification) and yield a “processed” cartridge containing a final nucleic acid product (e.g., an mRNA-lipid formulation). Next, the processed cartridge may be transferred to a finishing instrument, which may establish sterile access, extract and analyze the product, execute fill/finish operations, and thereby produce a finalized therapeutic as well as a cartridge that is “finished” for disposal. By organizing the workflow around these discrete cartridge states, each instrument may focus on specialized operations while maintaining a coherent, closed process under the system's unified control.

The system may be particularly suited for providing mRNA therapeutics, which may require DNA amplification, in vitro transcription, purification, and encapsulation in lipid nanoparticles or other carriers. Each of these steps may be executed within a cartridge, which may be disposable, via a single processing instrument. Specifically, the processing instrument's actuating modules may be programmed to actuate fluid mixing, temperature control, and other processing steps via corresponding modules of the cartridge. Because each cartridge may engage with a single processing instrument for manufacturing, cross-contamination may be minimized, and process variability may be greatly reduced compared to conventional systems. Furthermore, the system's modular nature may enable high-throughput small-batch runs, which is essential for personalized treatments such as individualized mRNA neoantigen vaccines for cancer. Rapid transitions between different patient-specific mRNA sequences may be accomplished by simply switching out cartridges, allowing the system to adapt quickly to diverse needs. For example, optional robotic arms or similar handling devices may be employed to automatically transfer cartridges among the pre-processing, processing, and finishing instruments while maintaining sterility within the system. Moreover, the system may further include a control interface for coordinating processing parameters, collecting data, and maintaining records for regulatory compliance.

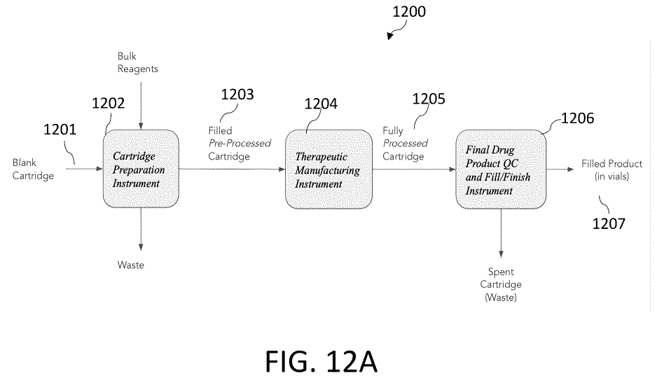

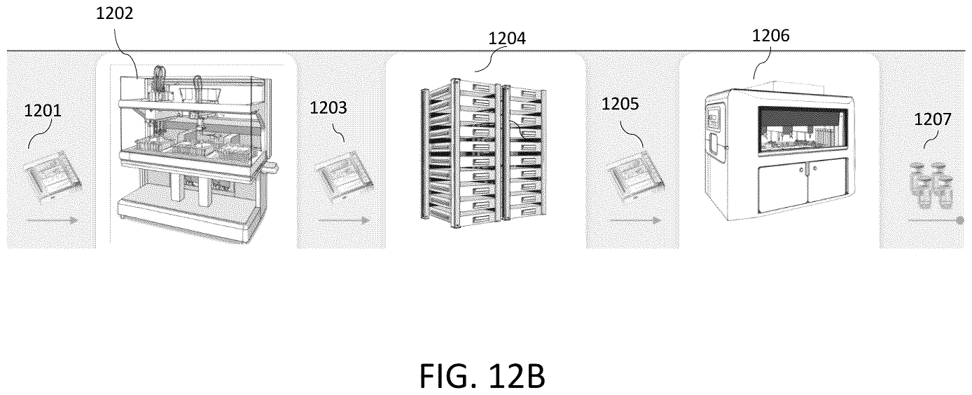

The system may comprise a control system to control an overall processing workflow for each product. The workflow may generally be streamlined bioprocessing sequence involving cartridge preparation, therapeutic manufacturing, and final quality control with fill/finish. An exemplary such process is shown in FIGS. 12A-12B, which will be described in detail herein. Briefly, the process may begin with preparing a blank cartridge in a first instrument that fills it with essential reagents, such as cell culture media, buffers, and reaction components. Once filled, the cartridge may be sealed, verified for accuracy, and made ready for processing. In the next step, the loaded cartridge may be transferred to a second instrument that carries out the actual bioprocessing steps, which may include cell culturing, genetic modification, protein expression, or other specialized manufacturing techniques. Finally, the processed cartridge may be moved to a third instrument that manages sterile fluid transfer into product vials or containers under controlled conditions. At this stage, the product may undergo quality control checks before it is either shipped or prepared for immediate clinical use. Once the product is finalized, the cartridge may be disposed of. The control system may be configured to oversee each step of this workflow.

In some variations, the control system may be configured to generate and maintain an electronic batch record (EBR) that records data at each stage of cartridge preparation, processing, and finishing. This EBR may incorporate patient-specific manufacturing parameters, reagent usage, process measurements (e.g., temperature, pressure, optical signals), and any events or operator interactions. The control system may continuously update the EBR, linking all relevant data to a unique cartridge identifier, thereby creating a unified, traceable history of product evolution from initial blank cartridge to final therapeutic product. In some variations, the EBR may include time-stamped records of each step, real-time sensor feedback, and quality control results, providing comprehensive documentation that may comply with regulatory requirements (such as 21 CFR Part 11) and facilitating efficient review, audit, and approval processes.

The three-instrument system may provide several advantages over existing manufacturing approaches. First, the integration of cartridge preparation, processing, and finishing into a unified workflow may reduce contamination risks compared to traditional open manufacturing approaches. Second, the rack-mounted, modular system configuration for the processing stage may enable parallel processing of multiple patient-specific therapeutics within a compact footprint, significantly increasing manufacturing efficiency while reducing facility costs. Third, the closed-system approach may maintain product sterility throughout the process without requiring manual interventions, thereby ensuring consistent product quality while minimizing operator requirements. Fourth, the system's ability to prepare cartridges just-in-time may reduce reagent waste and improve therapeutic effectiveness by optimizing reagent stability. Finally, the scale-out architecture may enable gradual capacity expansion aligned with patient population needs, providing flexibility not available in traditional fixed-capacity manufacturing facilities.

By consolidating the various stages of nucleic acid manufacturing—cartridge preparation, nucleic acid processing, and product extraction/finalization—into a single integrated workflow, the present invention may achieve high-throughput production of “lot of one” therapeutics that satisfy the stringent needs of personalized medicine. Additionally, because each step of the workflow may be continuously logged into a unified electronic batch record, the system may maintain robust traceability and regulatory compliance throughout the entire production cycle. Compared to the state of the art, which relies heavily on manual intervention and large-batch methods unsuited to small-volume output, the system may drastically shorten production timelines, reduce potential errors, and offer a more economical, scalable approach to manufacturing advanced nucleic acid therapeutics. As research and demand continue to expand in mRNA-based therapies, the disclosed platform may provide a versatile, compact, and automated model for meeting these emerging clinical and commercial requirements.

While the following description focuses on instruments and cartridges sized and shaped for small-batch manufacturing, it should be understood that variations of these components may be employed to accommodate different processing needs. For example, the systems herein may comprise instruments and cartridges of various sizes to manufacture therapeutics ranging from personalized, patient-specific formulations (e.g., individualized mRNA treatments) to relatively larger batches for treating a plurality of patients with the same therapeutic.

As used herein, the terms “module” and “actuating module” refer to a set of one or more components or tools of a device (e.g., a cartridge or a processing instrument) that may be used to perform a bioprocessing operation. In some variations, the modules and/or actuating modules herein may be reconfigurable. In some variations, the modules and/or actuating modules herein may be used to perform more than one bioprocessing operation.

As used herein, the terms “material,” “biomaterial,” “genetic material,” “sample,” and “product” refer to an initial, intermediate, or final manufactured therapeutic. The material may include one or more biomolecules, such as proteins, lipids, nucleic acids, carbohydrates, and combinations thereof. For mRNA processing, material may include nucleic acid-based material, such as one or more of DNA, a DNA template, and RNA (e.g., crude mRNA, unpurified and/or purified mRNA), including portions thereof. Additionally, or alternatively, in some variations, the material may include tRNA, rRNA, taRNA, derivatives thereof, and/or combinations thereof. As discussed throughout, the material may be carried and processed by a cartridge throughout a bioprocessing workflow. In some variations, one or more samples of the material may be taken from the cartridge and analyzed. Such samples may or may not be returned to the cartridge following analysis.

As used herein, with respect to a final therapeutic product, “small volume” and “small batch” refer to a per-patient dose of about 0.05 mL to about 50 mL (e.g., about 5 mL to about 30 mL, or about 15 mL to about 25 mL per patient dose), and/or a total treatment dose of about 0.5 mL to about 200 mL (e.g., about 1 mL to about 100 mL for a complete treatment course). With respect to bulk mRNA (before LNP encapsulation) within the product, “small volume” and “small batch” products may contain about 1 μg to about 20 mg of mRNA (e.g., about 5 μg to about 10 mg), while the concentration of mRNA may range from about 0.01 mg/mL to about 20 mg/ml (e.g., about 0.05 mg/mL to about 10 mg/mL).

Although the following description may focus on small-volume production, it may also be adapted for larger runs using the same principles of automation and integration. No matter the batch size, the system's reliance on an enclosed, automated workflow may maintain sterility and consistency while decreasing labor requirements and opportunities for human error.

Detailed exemplary variations of these systems, devices, methods, and aspects thereof are elaborated below.

### Automated Bioprocessing System

The automated bioprocessing system may be configured to manufacture a nucleic acid product contained in a cartridge. The system may comprise one or more of each of a pre-processing instrument, a processing instrument, and a post-processing instrument. Each instrument may be configured to perform one or more preparation, manufacturing, or finishing steps on the nucleic acid product by engaging the cartridge. Each instrument may be modular and positioned within a larger enclosure of the system. Some or all of the instruments, such as a plurality of processing instruments of the system, may be arranged in a scalable, adjustable configuration to support a plurality of simultaneous, small-volume manufacturing runs resulting in reliable, small-batch therapeutics for biomedical applications. In some ways, the system may be conceptualized as a data center architecture adapted for bioprocessing, where a plurality of independent instruments operate in parallel, using a plurality of corresponding cartridges, within an optimized spatial arrangement.

The system may comprise a support unit configured to house an array of instruments, as well as a control system (e.g., one or more controllers) communicably coupled to each of the instruments and configured to coordinate operations, manage data, and oversee processing. The control system may additionally generate an updated batch record for a cartridge/nucleic acid product at any point during pre-processing, processing, or post-processing operations. The system may produce nucleic acid therapeutics, such as mRNA vaccines, using templates of synthesized DNA and/or DNA sampled from one or more patients, which may generally be contained in the cartridge through most or an entire duration of a processing workflow.

In some variations, the cartridge may comprise a disposable (e.g., single use), self-contained unit. The cartridge may be configured to maintain a closed environment when engaged with an instrument to maintain product sterility, reduce workflow complexity, and limit batch tracking requirements of the system. To achieve this, the cartridge may be preloaded, by the pre-processing instrument, with all fluids and materials necessary to complete the workflow within the processing instrument and without requiring transport to another processing instrument or part of the system.

To execute the workflow, the processing instrument may comprise one or more actuating modules configured to act on one or more corresponding modules of the cartridge. In some variations, components of the cartridge modules and/or processing instrument actuating modules may be updated, removed, or replaced to provide a uniquely configured cartridge and/or processing instrument. To this end, highly customized bioprocessing workflows may be achieved using the automated bioprocessing systems and devices herein.

In some variations, the cartridges herein may be provided in same or varying sizes depending on a batch volume. A first cartridge having a total fluid capacity on the order of microliters (μL) to milliliters (mL) may be configured to process small batches of therapeutics. Such a cartridge may be useful in processing personalized therapeutics (e.g., for cancer treatments) for a single patient or a small group of patients (e.g., fewer than 3 patients, fewer than 5 patients, fewer than 10 patients, fewer than 15 patients, or fewer than 25 patients). In some variations, the system may comprise a plurality of first cartridges and may only comprise the first cartridges in some cases. In alternative variations, the system may further comprise a second cartridge having a total fluid capacity on the order of liters (L) configured to process relatively larger batches of therapeutics, such as for vaccine production for many individuals (e.g., more than 50 patients, more than 100 patients, more than 500 patients, more than 1,000 patients, more than 2,500 patients, or more than 5,000 patients). Accordingly, the first cartridge may be smaller than the second cartridge in one or more dimensions. The instruments may also be provided in various sizes (e.g., a first size and a second, larger size) to enable bioprocessing using various cartridges. In some variations, the systems herein may include a first set of one or more cartridges having a first fluid capacity and a second set of one or more cartridges having a second, larger fluid capacity. The systems may additionally include a first set of one or more instruments (e.g., processing instruments) configured to interface with the first set of cartridges, and a second, larger (in size height, width, and/or length) set of one or more instruments (e.g., processing instruments) configured to interface with the second set of cartridges.

Moreover, the system enclosure may comprise a room or area at least partially surrounding or housing one or more instruments and/or one or more support units that carry a plurality of instruments therein. In some variations, the system may comprise a multi-tiered arrangement of support units organized in a hierarchical structure defined by: a primary level comprising individual, modular instruments, a secondary level comprising a support unit containing a plurality of instruments therein, a tertiary level comprising a plurality of support units arranged in clusters, and a quaternary level comprising a plurality of clusters arranged in an array or other pattern within the system enclosure (e.g., a manufacturing facility). This architecture may enable each instrument included in the configurable arrangement to operate independently and in parallel, while the system as a whole remains integrated from a control, data-logging, and materials-handling standpoint. Thus, the hierarchical system architecture may facilitate scalable, high-throughput manufacturing of nucleic acid material with efficient use of available facility space.

Further, the system may optionally include a robotic transport system comprising one or more robots or robotic arms configured to transfer cartridges between instrument classes. The robotic transport system may be communicably coupled with the control system and receive instructions therefrom for transferring cartridges about the system. The robotic transport system may thus further reduce the system's requirement for manual operations.

Patient-specific mRNA therapeutics and may be manufactured with minimal operator intervention, often within about 5 hours to about 15 hours of start time (e.g., within about 6 to about 14 hrs, about 7 to about 13 hrs, about 8 to about 12 hrs, about 9 to about 11 hrs, or about 10 hrs).

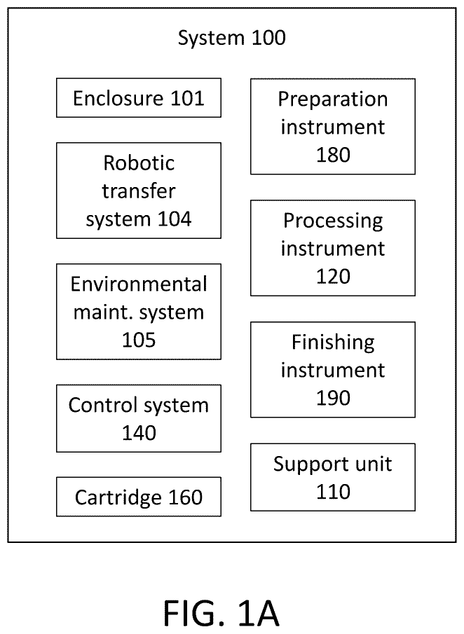

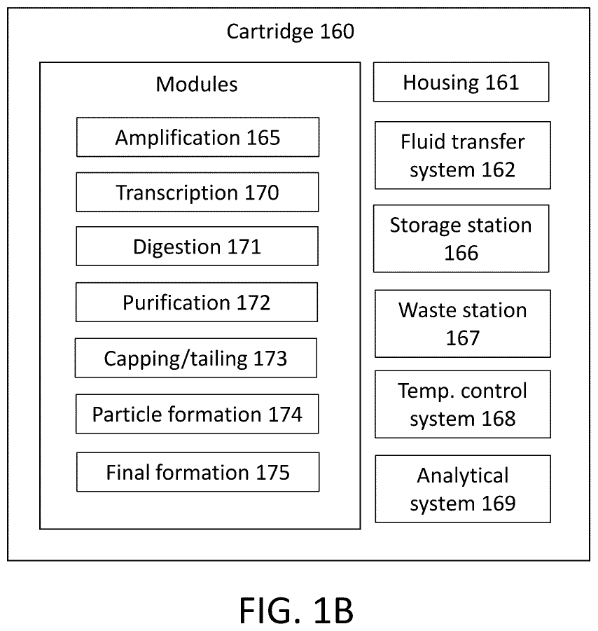

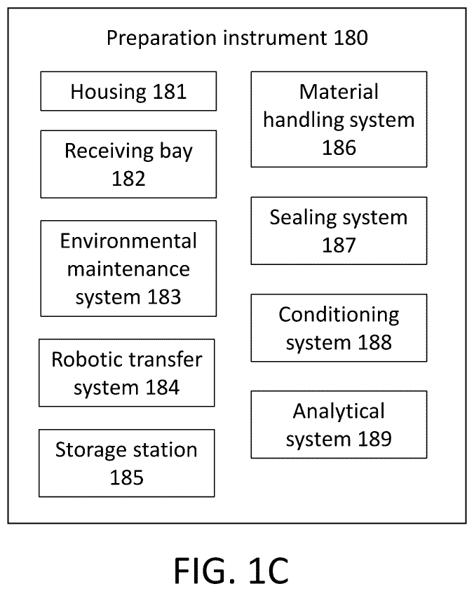

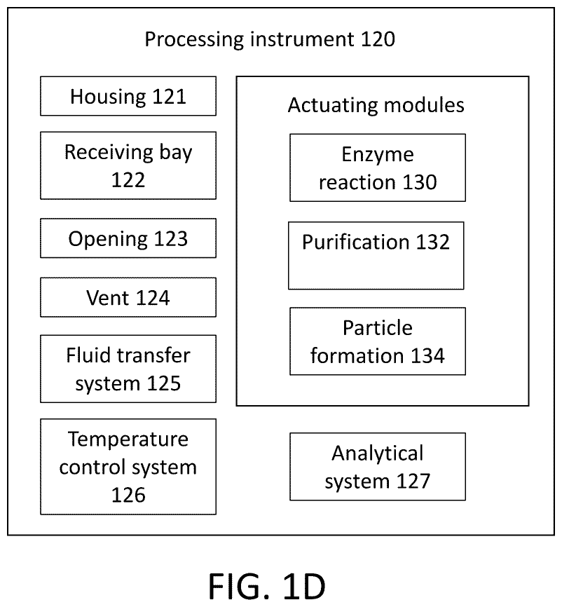

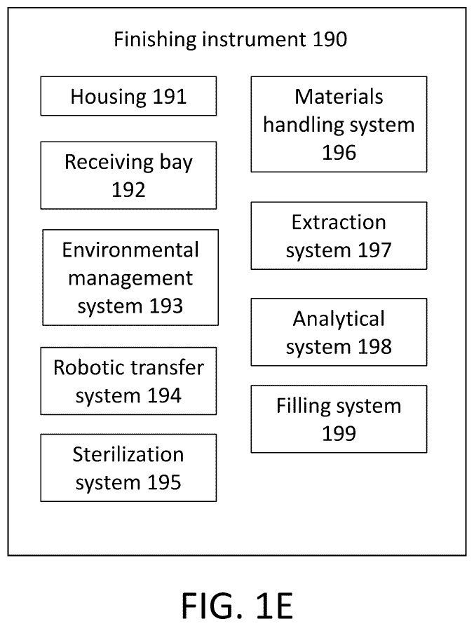

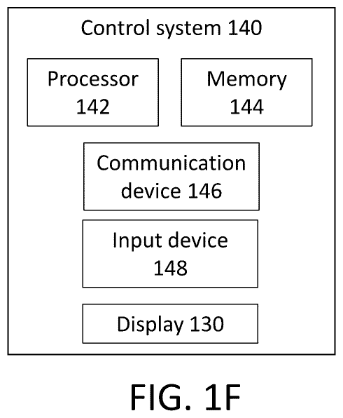

FIG. 1A is a block diagram of an illustrative variation of an automated bioprocessing system (“system”) 100 comprising an enclosure 101, a support unit 110, a preparation instrument 180, a processing instrument 120, a finishing instrument 190, a cartridge 160, a control system 140, and optionally a robotic transport system (RTS) 104 and/or an environmental maintenance system (EMS) 105. The system 100 may produce therapeutic products within about 5 hours to about 15 hours of a workflow start time, such as within about 6 to about 14 hrs, about 7 to about 13 hrs, about 8 to about 12 hrs, about 9 to about 11 hrs of the start time, or about 10 hrs following the start time.

The enclosure 101 may comprise a fully or partially enclosed area. A footprint of the system 100 may be defined by a perimeter of the enclosure 101. The enclosure may contain the remaining system components therein. In some variations, the enclosure 101 may comprise one or more rooms, such as within a warehouse or manufacturing facility.

The enclosure 101 may be configured to meet International Organization for Standardization (ISO) standard ISO8 or better (e.g., ISO7, ISO6, ISO %, or cleaner). In some variations, the system 100 may optionally comprise the environmental maintenance system (EMS) 105, which may be integrated with the enclosure 101. For example, EMS 105 may include one or more sensors configured to monitor environmental conditions of the interior of the enclosure 101. These sensors may be distributed throughout the enclosure 101 and may be configured to detect, for example, temperature, humidity, particle counts, and particle concentrations. The sensors may be configured to collect data continuously, periodically, and/or upon request (e.g., by the control system 140). The EMS 105 may further include one or more actuators configured to adjust environmental conditions within the enclosure 101, such as a humidity control assembly (e.g., humidifier or dehumidifier), airflow regulators (e.g., variable-speed fans, blowers, and dampers), and additional filtration units or recirculating ventilation subsystems. In some variations, the EMS 105 may comprise an actuator system, such as an HVAC system with a set of controllable elements (e.g., fans for airflow, dampers for directing air, heating/cooling coils for temperature control). Each sensor may be coupled to the control system 140 (e.g., a central controller thereof) such that sensor detections may be part of a feedback loop in which the control system 140 compares the data to criteria (e.g., ranges of acceptable values) and adjusts the environmental conditions when the data does not meet the criteria.

Moreover, in some variations, the enclosure 101 may include one or more access points, such as a door and/or a feedthrough, which may be positioned within a sidewall of the enclosure 101. In some variations, a sterilization procedure may occur within the access points of the enclosure 101. Such access points may be manually accessible and/or accessibly via the RTS 104.

The optional RTS 104 may be configured to move cartridges through the enclosure 101. The RTS 104 may include one or more robots which may be communicably coupled to and actuated by the control system 140. For example, the RTS 104 may be configured to transfer: a blank cartridge (i.e., an unused cartridge holding no reagents) to a preparation instrument, a filled/prepared cartridge (i.e., the same cartridge, but loaded and prepared for processing) from the preparation instrument to a processing instrument, a processed cartridge (i.e., the same cartridge, but storing a final product) from the processing instrument to a finishing instrument, and a filled product (i.e., an analyzed and re-packaged product of the final product) from the finishing instrument to out of the system 100. In some variations, one or more robots of the RTS may be configured to move throughout the enclosure 101. For example, first and second robots may be coupled to a same or different track, rail, or movable base (e.g., comprising wheels) within the enclosure 101, such as to one or more linear rails positioned centrally (e.g., on a floor or ceiling) within the enclosure 101, to enable movement of the first and second robots. Additionally, or alternatively, a second robot may be configured to move freely about the enclosure 101. In some variations, one or both of the first and second robots may be coupled to a movable base that is not fixed to another component of the system 100.

Each robot of the RTS 104 may include one or more robotic arms, such as one, two, at least one, at least two, or more than two robotic arms. In some variations, the robot may comprise an arm with multiple degrees of freedom (DOFs) (e.g., five, six, or more) and may be translatable along at least one axis and/or rotatable around at least one axis. In variations where a robot is coupled to a track or other movable base, the coupling may provide an additional (e.g., seventh) DOF. In some variations, the first robotic arm and the second robotic arm may share the same number of DOFs, while in other variations, the first robotic arm and the second robotic arm have a different number of DOFs to accommodate different functions. A robotic arm may comprise one or more pivot joints (e.g., one, two, three, more than three, etc.) around which at least a portion of the robotic arm may rotate. In some variations, the robotic arm(s) may comprise a plurality of articulating joints. Further, each robotic arm may comprise one or more end effectors configured to precisely handle the cartridge 160. For example, the second robot may comprise a movable base (e.g., comprising wheels) that is not fixed to another component of the system 100. A robotic arm may comprise one or more pivot joints (e.g., one, two, three, more than three, etc.) around which at least a portion of the robotic arm may rotate. In some variations, the robotic arm(s) may comprise a plurality of articulating joints. Further, each robotic arm may comprise one or more end effectors configured to precisely handle the cartridge 160. For example, a robotic end effector may be configured for one or more of: actuating instrument receiving bays between open and closed configurations, accessing an interior of a cartridge (when the cartridge is not engaged with an instrument), facilitating engagement and/or disengagement between a cartridge and an instrument, actuating openings (e.g., feedthroughs) of the enclosure between open and closed configurations, grasping one or more cartridges at once, and the like. In some variations, an end effector may be configured to detect batch data from an RFID or similar label on the cartridge housing. Additionally, or alternatively, in some variations, the end effector may be temperature-controlled for handling of cartridges comprising sensitive reagents. In some variations, a first robotic arm and a second robotic arm may act in coordination and may be orchestrated to perform coordinated motions. In some variations, the RTS 104 may comprise a conveyor for transporting one or more cartridges about the enclosure 101.

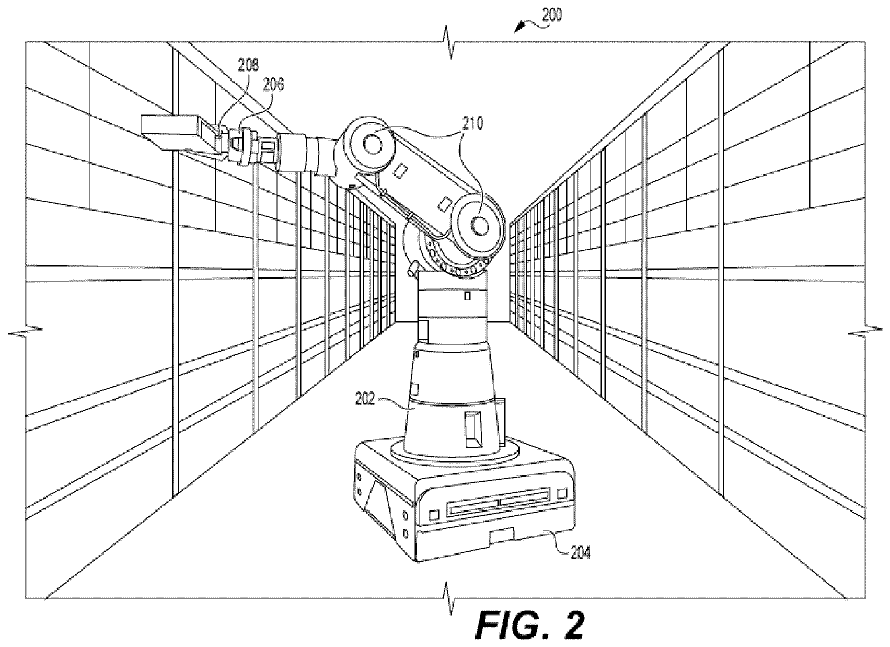

A variation of an RTS robot is shown in FIG. 2. Specifically, FIG. 2 depicts a view of exemplary variation of an automated bioprocessing system 200. As shown, the system 200 may comprise an interior zone through which a robot 202 may be provided. The robot 202 may comprise a moveable first end 204 enabling the robot 202 to move throughout the system 200. Additionally, the robot 202 may comprise a second end 206 comprising an end effector 208. The end effector 208 may be configured to support a cartridge during transport operations (e.g., from instrument to instrument). The robot 202 may also comprise a plurality of joints 210 that configure the robot 202 as an articulating robotic arm. The robot 202 may thus be translatable and rotatable with multiple degrees of freedom.

### I. Cartridge

The cartridge may be a fully integrated and self-contained device configured to enable automated production of individualized therapeutics, such as mRNA products. By housing all necessary fluids, materials, and components (for instance, reaction chambers, microfluidic pathways, mixers, and sensors) within a closed, single-use enclosure, the cartridge may be transported among the three classes of instruments without requiring repeated fluid transfers or external handling. Specifically, the cartridge may be introduced as a blank cartridge into the preparation instrument, filled with reagents and sealed, then moved to the processing instrument for nucleic acid production, and finally transferred to the finishing instrument to extract the product. This closed workflow may preserve sterility and reduce contamination risks across all stages.

FIG. 1B is a block diagram of an exemplary variation of a cartridge of the automated bioprocessing system of FIG. 1A. As shown, the cartridge 160 may comprise a housing 161, one or more modules 165 and 170-175, described in detail below, and one or more operating systems 162 and 166-169, also described in detail below.

The housing 161 may be designed to maintain a closed environment throughout processing. In some variations, the housing 161 may be sealable to enclose an internal space containing all modules, systems, and stations therein. This design may preserve sterility from start to finish while still allowing alignment features, fluidic ports, thermal contact surfaces, and electrical connectors to pass through sidewalls in precisely defined locations.

In some variations, the housing 161 may comprise a first (e.g., top) portion and a second (e.g., bottom) portion configured to be coupled together to form a sealed environment. The first portion may comprise a substantially planar upper surface with downwardly extending sidewalls, while the second portion may comprise a base with upwardly extending peripheral walls, such that when assembled, the respective walls overlap or interlock. In some variations, this overlap or interlock may create a sealed junction between the first and second portions. In some variations, this junction may be secured through various coupling mechanisms, such as via thermal elements, pressure applicators, snap-fit connections, compression fittings, threaded interfaces, or ultrasonic welding.

In some variations, the interface between the top portion and bottom portion may incorporate a gasket or O-ring positioned within a corresponding groove or channel to enhance the seal integrity. The gasket may be composed of elastomeric material capable of maintaining compression while resisting chemical degradation from reagents stored within the cartridge. Alternatively, the interface may be designed to create a hermetic seal through direct material-to-material contact. In this case, the seal may form via thermal or ultrasonic welding processes that fuse the first and second portions together along their corresponding peripheral edges.

Additionally, or alternatively, the housing 161 may comprise at least one fluid portion (e.g., a plurality of fluid ports) configured to provide selective access to a fluid compartment within the cartridge. Each fluid port may be individually sealable after its corresponding fluid compartment is loaded with a reagent or other material. Similarly, the fluid ports may be sealed through the application of localized heat and/or pressure, as explained in detail below with respect to the preparation instrument 180.

Moreover, the housing 161 may comprise a label or identifier, such as an RFID tag or barcode, may be attached to an exterior surface of the housing 161 so that the control system 140 may verify the cartridge's batch number, reagent status, type, and workflow throughout preparation, processing, and finishing. For example, each of the instruments 120, 180, and 190 may be configured (e.g., using scanners) to detect the label on the cartridge 160. Because the cartridge 160 may remain fully enclosed during operation, and may be disposed of or recycled afterward, it may be well suited for GMP-compliant workflows that require controlled, sterile conditions and tight process traceability.

The housing 161 may comprise a square, rectangular, substantially square, substantially rectangular, or rounded shape (e.g., a substantially square or rectangular cross-sectional shape). In some variations, the housing 161 may be sized and shaped in a rectangular volume, similar to a VHS-type tape or cassette, thereby allowing for convenient handling and storage. For example, the cartridge housing 161 may measure about 6 in to about 8 in (about 15 cm to about 20 cm) in length, about 3.5 in to about 5 in (about 9 cm to about 13 cm) in width, and about 1 in to about 2 in (about 2.5 cm to about 5 cm) in thickness, though other size ranges may be possible. This form factor may be sufficiently compact to allow for a plurality (e.g., tens or dozens) of cartridges may be loaded into a processing instrument-based support unit or storage area without difficulty.

The housing 161 may be constructed of USP Class VI-certified materials and manufactured under ISO 13485 and GMP-compliant processes, ensuring biocompatibility and regulatory alignment. All fluid-contacting surfaces of the cartridge 160, including tubing, fluid compartments, and membranes, may either be presterilized or constructed of materials suitable for sterile assembly, thereby enabling an aseptic bioprocess from DNA to final mRNA product. The housing 161 may be formed using a variety of fabrication methods (e.g., molding, machining, extruding, or 3D printing) and sealed into a disposable format that helps reduce cross-contamination and operator intervention.

The housing 161 may additionally comprise one or more connectors/interfaces for engaging an instrument (e.g., the processing instrument 120). For example, the housing 161 may comprise one or more mechanical connectors extending from its external surfaces for coupling to corresponding instrument connectors and maintaining a fixed position of the cartridge 160 when engaged with the instrument. Additionally, the housing 161 may comprise one or more fluidic, thermal, electrical, and/or sensor interfaces that align with corresponding instrument elements and interfaces, such as with the processing instrument connectors when the cartridge 160 is within its receiving bay.

Within the housing 161, the cartridge 160 may carry a series of interconnected modules, each comprising one or more fluid compartments (e.g., reaction chambers, storage containers) used for a particular step in the nucleic acid manufacturing workflow. These steps may include amplification (e.g., PCR), magnetic bead purification, in vitro transcription, tangential flow filtration, and LNP formulation. Each fluid compartment may comprise components enabling it to be specifically optimized for its function (e.g., enhanced thermal conductivity walls for rapid PCR cycling, magnetically enabled chambers for bead separations, microfluidic channels for lipid particle formation, etc.). In some variations, at least a portion of one or more fluid compartments within the cartridge 140 may be optically transparent or translucent to enable sensors on the instrument side (e.g., on one or more of the instruments 120, 180, and 190) to detect process parameters, such as a fluid level within a given fluid compartment. The cartridge 160 may also comprise one or more operating systems configured to support the processing requirements of the modules. The operating systems may support the modules by maintaining critical process parameters and performing essential background tasks. In this way, each module may rely on one or more of these systems to carry out or verify a specific step of the workflow. The cartridge 160 may also incorporate stations for storing reagents, waste, intermediate products, and/or final products at various stages of processing. These systems and stations may, for instance, control fluid flow, maintain temperature, store reagents, and collect waste, thereby enabling the modules to complete the primary bioprocessing functions (e.g., amplification, transcription, digestion, capping & tailing, particle formation, purification, and formulation). As described in greater detail below, such operating systems and stations may include a fluid transfer system 162, a storage station 166, a waste station 167, temperature control system 168, and an analytical system 169.

### Fluid Transfer System (FTS)

The fluid transfer system (FTS) 162 of the cartridge 160 may fluidically couple the modules and stations within the cartridge 160 with a network of conduits configured to direct material throughout the cartridge under instrument control while keeping the entire process closed and sterile. The conduits (e.g. tubing or channels) may be coupled to valves (e.g., automated valves) and actuated by pump segments to guide the nucleic acid material, reagents, and wastes between modules and stations. For example, the FTS 162 may route fluids among the modules via one or more pump segments and valves accessible by the processing instrument 120 for driving fluid flow. In some variations, one or more fluid ports on the cartridge 160 may be configured to engage one or more corresponding fluid lines of the instrument 120 such that the cartridge 160 may receive bursts of compressed air or inert gas to push fluids through the fluidic pathways of the FTS 162. Moreover, the FTS 162 may connect one or more of the modules to a storage station 166 and/or a waste station 167.

### Storage Station and Waste Station

The storage station 166 may be configured to store reagents for use during a bioprocessing workflow. In some variations, reagents may be preloaded into the storage system 166 such that the cartridge 160 may be a self-contained cartridge device enabling efficient processing via a single processing instrument 120. The storage station 166 may include one or more fluid compartments, such as a plurality thereof, for storing different reagents and for providing varying storage conditions for the reagents. In some variations, the storage system 166 may comprise 1 to 20 fluid compartments, such as 2 to 15, 3 to 14, 4 to 13, 5 to 12, 6 to 11, 7 to 10, or 8 to 9 fluid compartments, including all ranges and subranges therebetween (e.g., 5, 6, 7, 8, 9, or 10 fluid compartments). Fluid compartments may comprise one or more temperature-controlled compartments for maintaining a range of temperatures (e.g. about −20° C. about 2° C. to about 8° C., about room temperature) for storing enzymes, buffers, and lipids until needed.

One or more fluid compartments of the storage station 166 may advantageously be temperature-controlled by the TCS 168 (e.g., via an instrument TCS). For example, a fluid compartment of the storage station 166 may interface with one or more thermal elements to control a temperature within the fluid compartment. In some variations, one or more fluid compartments within the storage station 166 may be maintained at about 2° C. to about 8° C., such as at about 4° C. Additionally, or alternatively, one or more fluid compartments may be maintained at freezing or below freezing temperatures, such as within about −40° C. to about 0 ° C., or at about −20° C. Accordingly, the storage station 166 may provide a suitable environment for one or more reagent types, including enzymes, buffers, and/or lipids used throughout RNA processing.

In some variations, one or more such fluid compartments of the storage station 166 may be loaded (and the cartridge 160 sealed) by the preparation instrument 180 prior to loading the cartridge 160 within the processing instrument 120. This may enable the cartridge to maintain a closed internal environment throughout some or all of a nucleic acid material processing workflow by reducing or eliminating the need for transferring fluids to or from the cartridge using other devices.

In some variations, the storage station may comprise a fluid compartment configured to receive a nucleic acid product, such as a final mRNA-lipid product from the particle formation module.

Like the storage station 166, the waste station 167 may include one or more fluid compartments for storing wastes. For example, the waste station 167 may be configured to collect reagents, byproducts, or other waste fluids generated by the cartridge modules. In some variations, one or more segregated fluid compartments (e.g., for aqueous versus organic waste) may be incorporated, each optionally sized to match the maximum anticipated process volume. The FTS 162 may fluidically couple each module to at least one fluid compartment of the waste station 167.

In some variations, one or more sensors (e.g., bubble sensor(s), flow sensor(s), pressure sensor(s), and/or camera(s)) of the cartridge 160 and/or an interfacing instrument may be configured to detect a fluid level and/or flow rate within the storage station 166 and/or waste station 167 (e.g., within one or more fluid compartments thereof) to prevent overflow therein. The sensors may be part of a cartridge analytical system 169 and/or of the corresponding instrument. In some variations, the fluid compartments of the storage station 166 and/or waste station 167 may be constructed of a partially or fully translucent or transparent material to enable such monitoring.

Moreover, one or both of the storage station 166 and waste station 167 may comprise a plurality of fluid compartments of the same or various sizes. Each fluid compartment may have a fluid capacity of about 10 mL to about 300 mL, such as about 15 mL to about 200 mL, about 20 mL to about 100 mL, or about 25 mL to about 75 mL, such as about 200 mL. The storage station 166 may be configured to store fluids at smaller volumes of, for example, about 1 μL to about 50 mL, such as about 100 μL to about 40 mL, about 500 μL to about 30 mL, about 1 mL to about 25 mL, about 5 mL to about 20 mL, or about 10 mL to about 15 mL (e.g., about 285 μL, about 9 mL, about 18 mL). A total fluid capacity of the storage station 166 and/or the waste station 167 may be about 1 mL to about 5 L, such as about 10 mL to about 3 L, about 50 mL to about 2 L, about 100 mL to about 1.5 L, about 250 mL to about 1 L, or about 500 mL to about 750 mL. In some variations, a fluid compartment of one or both of the storage and waste stations 166, 167 may comprise a storage capacity of about 0.25 L to about 2 L. In some variations, for personalized applications, a fluid compartment of one or both of the storage and waste stations 166, 167 may comprise a storage capacity of about 25 mL to about 40 mL (e.g., about 35 mL), or about 50 mL to about 75 mL (e.g., about 50 mL).

### Temperature Control System (TCS)

The TCS 168 may enable multi-zone temperature regulation within the cartridge 160 to protect and stabilize temperature-sensitive components, such as enzymes, buffers, or lipids. In some variations, each cartridge module may include at least one on-board temperature sensor that provides real-time feedback to the control system 140, allowing it to determine whether the module's measured temperature is within an acceptable range. If a temperature reading is out of range, the control system 140 may adjust the power applied to one or more thermal elements interfacing with the cartridge, typically via thermal elements of an instrument TCS (e.g., Peltier elements and/or fluid-cooled plates). The TCS 168 may include a primary liquid cooling circuit that serves as the thermal backbone, along with a cascade control architecture in which primary temperature sensors drive precise control of the thermal elements, while secondary sensors monitor process temperatures directly for redundancy. This dual-monitoring approach may help ensure an accuracy of about ±0.5° C. and enhances the reliability of continuous temperature maintenance.

In some variations, the cartridge 160 may incorporate multiple discrete temperature zones that could be maintained at different setpoints, for example about −20° C. for subzero storage, about 2 to about 8° C. for refrigerated storage, or about 30 to about 50° C. for incubation processes such as in vitro transcription. Internally, the cartridge 160 may include valves, gaskets, or insulating layers designed to sustain these distinct temperature conditions with minimal heat exchange between adjacent compartments. When the cartridge 160 is engaged with the processing instrument 120, the instrument-based TCS 126 may thermally couple to the cartridge housing—via direct contact surfaces or dedicated heat-exchange interfaces—thereby activating, adjusting, or supplementing the on-board thermal regulation features of the cartridge. In this manner, the TCS 168 may consistently maintain each region of the cartridge 160 at the required temperature for its respective nucleic acid manufacturing step.

### Analytical System

In some variations, an analytical system 169 may be integrated into the cartridge 160 to monitor product and/or process parameters in real-time (e.g., temperature, pressure, pH, conductivity, UV absorbance). For example, each module may include one or more sensors for feedback control by the processing instrument's controller. The cartridge 160 may thereby support fully automated quality control at each stage, with data logged electronically for GMP compliance.

The analytical system 169 may include one or more sensors for detection of process and/or product parameters at various points along the processing workflow. The sensor(s) may include one or more pH sensor(s), conductivity sensor(s), temperature sensor(s), pressure sensor(s), bubble sensor(s), and/or camera(s). The pH, conductivity, and temperature sensor(s) may be configured to provide real-time feedback on sample parameters, and the pH sensor(s) may be configured to provide real-time feedback on process parameters like fluid flow rate throughout the FTS 162, a pressure differential of tangential flow filtration, and the like. The bubble sensor(s) and/or camera(s) may provide real-time feedback on cartridge parameters like a fluid level within a module. As an example, the integrated analytics may include UV spectroscopy at a range of about 150 nm to about 300 nm, such as at about 230, 260, 280 nm wavelengths, conductivity measurements in the range of about 1 mS/cm to about 40 mS/cm, temperature sensors with ±0.5° C. accuracy, flow sensors measuring about 0 mL/min to about 20 mL/min, pressure sensors for about 0 psi to about 50 psi, and pH monitoring in the range of about 5.5 to about 9.0 (e.g., about 6 to about 8.5).

The analytical system 169 may be integrated via, for example, sensor leads or optical paths routed to instrument-side electronics through standardized connectors. This may ensure that the processing instrument's controller receives a continuous stream of data to confirm product quality and control process conditions dynamically. Such in-process analytical checks may be essential for GMP compliance, as the system may automatically log critical values (e.g., reaction temperature or flow rate) to a batch record.

Furthermore, the system 100 may be configured to adjust process parameters in real-time. For example, in some variations, one or more sensors of the analytical system 169, and/or one or more of the instrument analytical systems (e.g., processing instrument analytical system 127), may be part of a feedback loop whereby the system is configured to make adjustments based on calculated parameters. For example, in some variations, one or more of a pH, conductivity, temperature, pressure, and/or the like may be pre-set (e.g., by the controller 140) to be maintained within a particular range. If a sensor detects a parameter outside of the range, the control system 140 may be configured to (i) alert an operator, (ii) pause or cease functioning for a set period of time or indefinitely, and/or (iii) self-correct.

Referring now to the cartridge modules 165 and 170-175, these modules may be configured to engage with the processing instrument 120. Each module may be configured to perform a step (e.g., at least one step) of a bioprocessing workflow, such as of a nucleic acid material (e.g., RNA) processing workflow. Such a workflow may include one or more of each of an amplification step, a transcription step, a digestion step, a capping/tailing step, a purification step, a particle formation step, and a concentration step. In some variations, one or more of the modules herein may share some or all of a set of components. The cartridge 160 may be configured with all or fewer than all of the following modules.

### Amplification Module (AM)

The AM 165 may be configured for PCR amplification (e.g., DNA template multiplication) within a controlled thermal environment. It may comprise a reaction chamber having high thermal conductivity walls to facilitate rapid temperature cycling (e.g., about 60° C. to about 95° C.) driven by processing instrument-mounted actuators. One or more thermal elements of the TCS 126 may interface with the AM 165 to achieve heating rates of greater than about 5° C./sec and cooling rates of greater than about 3° C./sec, supporting about 22 to about 28 PCR cycles for optimal amplification. In some variations, real-time UV sensors and/or optical sensors may be integrated to monitor amplification progress. The PCR chamber may be configured for holding about 100 μL to about 50 mL of reaction fluid, with personalized applications typically using volumes of about 100 μL to about 200 μL.

### Transcription Module (TM)

The TM 170 may support in vitro transcription, in which amplified DNA templates, buffers, and enzymes are combined to produce an RNA transcript. It may include a thermally controlled reaction chamber and one or more magnetic or mechanical stirring components for homogeneous mixing. The processing instrument TCS 126 may engage the TM 170 to maintain setpoint temperatures within about 30° C. to about 50° C. (e.g., at about 37° C.) for optimal transcription kinetics. The reaction chamber may comprise an in vitro transcription (IVT) chamber sized for about 100 mL to 300 mL of material (such as about 200 mL), though for personalized “lot of 1” applications, the actual reaction volumes may be significantly smaller, such as around 100 μL to about 500 μL with components including DNA Template (e.g., about 100 μL), IVT MM1 (e.g., about 266.7 μL), IVT MM2 (e.g., about 33.3 μL), DNase Mix (e.g., about 5.5 μL), and Proteinase K (2.5 μL) as specified in the cartridge concept.

### Digestion Module (DM)

The DM 171 may be configured to digest or remove unwanted DNA after transcription. In some variations, the DM 171 may share a reaction chamber with the TM 170 or the AM 165. The digestion step may also be thermally controlled (e.g., maintained at 37° C.) and monitored using on-board sensors. The reaction chamber may comprise a capacity of about 100 mL to about 300 mL for DNase digestion or analogous post-transcription processing at about 37° C., though personalized applications may utilize smaller volumes consistent with IVT reaction volumes.

### Purification Module (PM)

The PM 172 may include one or more submodules for filtration or separation-based purification of RNA, such as a tangential flow filtration module (TFFM), magnetic separation module, and/or a chromatography module. In some variations, the PM 172 may include one or more conduits and membranes sized for small-batch mRNA purification. A conduit in the PM 172 may comprise tubing, such as a looped tube. In some variations, these conduits feature internal diameters ranging from about 0.5 mm to about 2.0 mm, optimized to maintain laminar flow characteristics while minimizing sample loss through non-specific binding. The conduits can be part of a network that is constructed from low-protein-binding materials such as modified PTFE or medical-grade silicone elastomers with surface treatments to reduce nucleic acid absorption. In some variations, the membrane systems can be scaled appropriately for the processing volumes typical in personalized medicine applications (e.g., about 5 to about 50 mL starting material in some variations). In some variations, the membranes can be hollow fiber TFF membranes with 300 kDa MWCO, offering about 0.01 m2 to about 0.05 m2 of active surface area while maintaining a relatively low internal volume (e.g., less than or equal 2 mL) to minimize product dilution and loss. In some variations, the membranes can comprise multi-layer filtration cassettes with hydrophilic modified polyethersulfone (mPES) or regenerated cellulose membranes, designed with channel heights of 100-250 micrometers to ensure uniform flow distribution across the entire membrane surface. Sample-retention pathways configured in serpentine or radial patterns are provided to maximize membrane utilization while maintaining processing pressures below about 60 psi (e.g., below about 50 psi, below about 40 psi, below about 30 psi, below about 20 psi, or below about 10 psi) to preserve mRNA integrity.

Pressure and flow sensors may be integrated to detect clogs or confirm correct flow rates during purification. The TFFM may additionally comprise feed, retentate, and permeate fluid compartments (each comprising a fluid capacity in the 100-700 mL range, such as in the 200-550 mL range) and an integrated filtration membrane area of, for example, about 0.01 to about 0.05 m². In some variations, the TFF membrane may comprise a 300 kDa MWCO specification. Magnetic selection or chromatography steps may also be performed within one or more fluid compartments of the PM 172, such as a flowcell of about 10 mL to about 100 ml capacity for bead-based separations or a 25-100 mL chromatography column with a loading volume around 700-800 mL and an elution collection capacity of about 200 to about 300 mL.

### Capping and Tailing Module (C&TM)

The C&TM 173 may be used to synthesize fully mature mRNA by adding a 5′ cap and a 3′ poly(A) tail. The C&TM 173 may comprise a reaction chamber configured to receive a sample and one or more reagents. In some variations, the capping and tailing steps may occur in a single reaction chamber shared with the digestion or transcription-based modules. The TCS 168 may maintain suitable temperatures for these enzymatic reactions, and optical or pH sensors may provide process feedback. The C&TM 173 reaction chamber may comprise a fluid capacity of about 100 to about 300 mL.

### Particle Formation Module (PFM)

The PFM 174 may be configured to encapsulate RNA in lipid nanoparticles (LNPs). It may include a microfluidic mixer, temperature-regulated mixing chamber, and/or stabilization chamber. For instance, the mixer may receive an RNA solution through one inlet and a lipid or ethanol-based solution through another, combining them at controlled flow rates to form mRNA-loaded LNPs. The mixture may then be rapidly cooled to maintain particle size and stability. As an example, the mixing chamber may comprise a capacity of about 50 mL to about 500 mL (e.g., about 100 mL), and the stabilization chamber may comprise a similar or larger capacity (e.g., up to about 400 mL). For personalized applications, the LNP formation process may operate at flow rates of about 0.01 to about 20 mL/min with a final lipid mix volume of about 10 mL.

### Final Formulation Module (FFM)

The FFM 175 may receive the formulated mRNA-lipid product to store, filter, and/or concentrate. In some variations, this module may comprise a fluid compartment, which may be a sealed reservoir, that is maintained within a temperature range (e.g., about 2° C. to about 8° C.) by the interaction between the TCS 168 and TCS 126, to preserve product integrity until the workflow is complete.

For personalized applications, the final product volume may be about 25 mL to about 40 mL. Moreover, a total fluid capacity of the cartridge 160 may be determined by a fluid capacity of each module and system therein having a fluid compartment(s). In some variations, the cartridge 160 may be configured to process a bioproduct for an individual patient or small group of individuals (e.g., about 100 individuals or fewer, under 50 individuals, under 25 individuals, under 10 individuals, under 5 individuals, or a single individual). In such variations, the cartridge may have a fluid capacity of about 100 μL to about a 100 mL, such as about 1 mL to about 75 mL, about 5 mL to about 50 mL, or about 10 mL to about 25 mL, including all ranges and subranges therebetween. Individual reaction chambers or modules within the cartridge may have smaller capacities, such as about 100 μL to about 500 μL for PCR and IVT reaction chambers, about 1 mL to about 20 mL for buffer storage compartments, and about 10 mL to about 50 mL for final product and waste capacity compartments.

The final product may be stored in a sealed reservoir maintained at a temperature (e.g., about 4° C.), and the control system 140 may notify the RTS 104 to retrieve the cartridge 160. The cartridge 160 may be removed in its entirety from the system 100, carrying the nucleic acid product and all waste materials therein, clearing the given processing instrument for the next run. Next, the cartridge 160 may be transferred to the finishing instrument 190, which may be configured to receive a finalized nucleic acid product from the cartridge 160, as will be described herein with respect to FIG. 1E. The cartridge 160, along with waste materials and/or remaining fluids from processing stored therein, may then be disposed of or recycled.

Thus, together, the cartridge features discussed above may enable a complete, automated, and closed workflow for small-batch or individualized nucleic acid therapeutics, providing GMP-compatible manufacturing in a compact, single-use format. By maintaining all reagents and process steps within one housing, the cartridge 160 may mitigate contamination risk, streamline materials tracking, and maintain product sterility throughout an entire workflow.

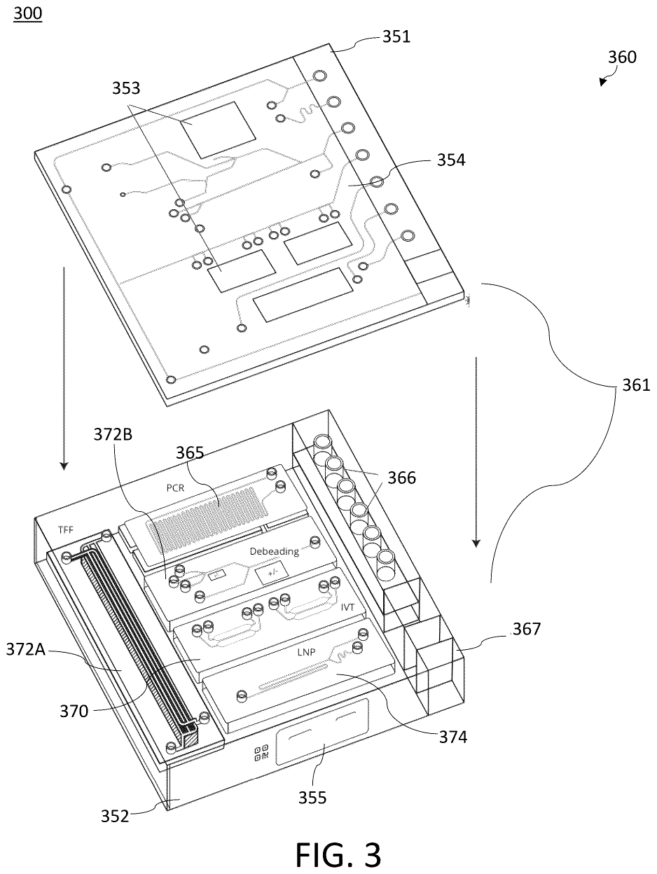

FIG. 3 provides an exploded view of a first portion of an automated system for bioprocessing 300. This first portion may comprise an exemplary configuration of a cartridge 360. The cartridge 360 may comprise a housing 361 comprising a first (top) portion 351 and a second (base) portion 352 that may be configured to be coupled together (e.g., sealed, such as via welding or heat/pressure application). The first portion 351 may comprise one or more observation windows 353 for enabling viewing into the cartridge's interior space, and may support a fluidic network 354 of the cartridge's FTS. The second portion 352 may hold the modules, which may include a purification module 372A configured for TFF, a second purification module 372B configured for magnetic separation, a transcription 370 module configured with a flow cell, and an amplification module 365 and particle formation module 374 each configured with thermally enhanced reaction chambers. Furthermore, the second portion 352 may comprise compartments making up a storage station 366 and waste station 367, as well as an interface 355 for releasably coupling to a transport robot.

### II. Preparation Instrument

The first class of instrument within the system may be a preparation instrument (e.g., the preparation instrument 180). The preparation instrument may perform the first step in a personalized therapeutics manufacturing workflow by transforming “blank” cartridges into patient-specific, reagent-loaded units ready for subsequent processing. As a result, the prepared cartridge may maintain a closed internal environment throughout some or all of (e.g., at least a portion of) nucleic acid material processing. This instrument may combine precision fluid handling, environmental control, and verification systems within a compact, self-contained enclosure providing all necessary functionality for precise, consistent, and accurate cartridge preparation. In some variations, the preparation instrument may be configured to prepare (“process”) one cartridge at a time. Alternatively, the preparation instrument may be configured to process a plurality of cartridges simultaneously.

A block diagram of an exemplary variation of the preparation instrument 180 is provided in FIG. 1C. The preparation instrument may generally comprise a housing 181, a receiving bay 182, an environmental management system (EMS) 183, a robotic transfer system (RTS) 184, a material handling system (MHS) 186, a sealing system 187, a conditioning system 188, and an analytical system 189.

The housing 181 may be configured to maintain a controlled environment therein. The housing 181 may include sidewalls that define an interior space and may fully or at least partially enclose the components of the preparation instrument 180. The housing 181 may comprise a rigid or semi-rigid frame constructed of materials suitable for maintaining the necessary environmental conditions within the enclosure. The external dimensions of the housing 181 may be configured to facilitate placement within a cleanroom or other controlled facility environment, while the internal dimensions may be optimized to accommodate the various operational systems of the preparation instrument 180. Additionally, the housing 181 may include one or more access panels or doors positioned at strategic locations to permit maintenance access while minimizing disruption to controlled environments.

The environmental management system (EMS) 183 may be integrated with the housing 181 and may comprise a filter, such as a HEPA filter, that extends across an upper portion of the interior space. The filter may be configured to direct filtered air into the interior space to maintain positive pressure and prevent contamination. The EMS 183 may create a precisely regulated environment exceeding cleanroom requirements through at least ISO8 (e.g., ISO 5/Grade A) filtered laminar airflow. In some variations, the airflow system may continuously sweep across the interior space to prevent particulate contamination. The EMS 183 may maintain a positive pressure differential of about 5 to about 20 Pa relative to the surrounding environment, employ an ordered flow pattern, and regulate temperature ranging from about 15 to about 30° C. with relative humidity of about 30% to about 70% RH. The EMS 183 may include temperature and humidity sensors distributed throughout the interior space, which may be coupled to the control system 140 to enable real time adjustment of environmental parameters. Further, real-time environmental monitoring may alert operators if conditions deviate from acceptable parameters, enabling immediate corrective action.

The preparation instrument 180 may be compartmentalized into several operational stations or zones, each dedicated to specific functions within the cartridge preparation workflow. The receiving bay 182 may be positioned within a sidewall of the housing 181. The receiving bay 182 may function as a controlled-access port configured to receive blank cartridges and subsequently output prepared cartridges. The receiving bay 182 may comprise a sealed chamber with interlocks that maintain environmental isolation during cartridge transfer operations. The physical dimensions of the receiving bay 182 may be configured to accommodate various cartridge designs, and its position within the housing 181 may facilitate efficient transfer of cartridges between external environments and the interior workspace. In some variations, an RFID or barcode reader (detector) may be positioned adjacent to the receiving bay 182 to authenticate each cartridge and initiate the patient-specific manufacturing process by retrieving the appropriate information from the control system 140.

The robotic transfer system (RTS) 184 may be configured to transport cartridges between various stations within the housing 181. The RTS 184 may be mounted on a structural framework that extends through central portions of the interior space, providing access to each operational station. In some variations, the RTS 184 may utilize grippers designed to securely handle cartridges. These grippers may be attached to one or more robots, such as one or more articulated arms or gantry systems capable of multi-axis movement throughout the interior workspace. In some variations, the RTS 184 may comprise an optical verification system comprising one or more cameras coupled with the control system 140 (e.g., to a preparation instrument-specific controller and/or to a general system controller). The optical verification system may be configured to verify cartridge identification and proper orientation throughout the cartridge preparation workflow. In some variations, the RTS 184 may continuously monitor cartridge positioning, ensuring consistent positioning throughout the operation process.

### Material Handling System (MHS) & Storage Station

The material handling system (MHS) 186 may function as the primary system for preparing reagents and loading them into each cartridge. The MHS 186 may receive a blank cartridge from the receiving bay 182 or the conditioning system 188 (as detailed below) via the RTS 184. In particular, the MHS 186 may include a preparation station and a handling system, and the RTS 184 may deliver the cartridge to the preparation station. The preparation station may comprise a housing configured to support the cartridge thereon, such as a substantially planar base plate. In some variations, the preparation station may comprise one or more alignment features or locks configured to couple to one or more corresponding components of a cartridge to hold the cartridge in a particular orientation and/or in place while the cartridge is loaded with reagents. The handling system may comprise a precision fluid handling system having access to the storage station 185. That is, the handling system may transfer fluids and materials from the storage station 185 to the preparation system.

The storage station 185 may be arranged along one side of the housing 181 and may comprise a plurality of temperature-controlled storage compartments configured to store a reagent. In some variations, a plurality of storage compartments may be arranged in an array configuration. Moreover, in some variations, each storage compartment may be configured to maintain temperatures between about −50° C. to about 50° C., depending on a reagent stored therein. For example, at least one storage compartment may be configured to maintain temperatures between about 2 to about 8° C. to preserve the activity of temperature-sensitive reagents. As another example, for specialized reagents, the storage station 185 may include dedicated deep-freeze storage compartments configured to maintain about −20° C. to about −80° C. conditions for enzymes and other highly temperature-sensitive materials. Each storage compartments may be couplable to the handling system through, for example, a transfer line. Each transfer line may be thermally insulated to maintain appropriate temperatures during reagent transfer.

The handling system may be positioned between the storage station 185 and the preparation station. The handling system may be configured to execute precise dispensing operations into the cartridge. In some variations, the handling system may comprise a dispenser or pipettor, such as a multi-channel pipettor. The handling system may be mounted to an interior surface of the housing 181, such as to an interior surface of a top or ceiling thereof. In some variations, the handling system may be mounted on a structural framework that allows for modular component replacement while maintaining system integrity. In some variations the handling system may be movable along a guiderail or track within the housing 181.

The handling system may be configured to handle volumes between about 0.01 μL and about 50 μL, such as about 0.5 μL and about 25 μL, about 1 μL and about 10 μL, or about 2.5 μL and about 7.5 μL. The handling system may be configured to deliver flow rates from about 0.1 μL/min to about 1000 μL/min. such as about 1 μL/min to about 750 μL/min, about 25 μL/min to about 500 μL/min, about 50 μL/min to about 250 μL/min, or about 75 μL/min to about 100 μL/min. Further, this system may provide a dispensing accuracy of ±1% for volumes greater than 10 μL and ±2% for smaller volumes between 1-10 μL, ensuring precise control of reagent ratios critical for subsequent nucleic acid manufacturing steps. To maintain sterility across cross-contamination between patient-specific formulations, the handling system may comprise one or more disposable components (e.g., pipettes) that are configured to be replaced between each cartridge preparation cycle.

The MHS 186 may be configured to execute sequential or parallel filling of a plurality of fluid compartments within the cartridge (e.g., within the storage station 166 of the cartridge 160). In some variations, the real-time monitoring of dispensing conditions occurs through multiple redundant methods, including pump displacement sensors, flow sensors, and weight verification. An automated air bubble detection and removal system may be integrated within the fluid pathways of the MHS 186, continuously monitoring the fluid path and identifying and eliminating air bubbles that could adversely affect dispensing accuracy. The MHS 186 may also incorporate inventory management hardware that maintains real-time tracking of reagent consumption and may provide continuous feedback on reagent availability and dispensing progress. Additionally, the MHS 186 may include compensation mechanisms that automatically adjust for environmental factors such as temperature and humidity that might affect dispensing accuracy, ensuring consistent results across varying conditions.

### Sealing System

The sealing system 187 may be positioned to receive cartridges from the MHS 186 via the RTS 184. The sealing system 187 may be configured to hermetically seal the cartridge. The sealing system 187 may comprise a housing comprising one or more thermal sealing elements and one or more pressure application mechanisms. At least one thermal sealing element and at least one pressure application mechanism may be used to seal a cartridge. In some variations, the sealing system 187 may employ a multi-zone thermal sealer with temperature control ranging from ambient to about 200° C. (+2.5° C.) and pressure regulation between about 0 N to about 100 N (±0.5 N) of force. The sealing temperature and pressure may be preset to result in proper seal formation without damaging cartridge components. In some variations, the sealing system 187 may additionally or alternatively incorporate a welder (E.g., an ultrasonic welder) to provide flexibility in accommodating various cartridge designs and materials. The welder may comprise transducers and anvils mounted within a same or different housing.

In some variations, the sealing system 187 may be configured to verify an integrity of the seal using one or more means. For example, an optical inspection system may be mounted within the sealing system 187 to examine seal uniformity and detect visible defects. Such a system may comprise one or more optical sensors coupled with the control system 140. As another example, a pressure decay testing system may verify a hermetic integrity of all sealed compartments. In some variations, the sealing system 187 may accommodate a plurality of seal test settings, allowing for adaptation to different cartridge geometries and configurations without requiring significant hardware modifications.

### Conditioning System

The conditioning system 188 may be configured to condition blank cartridges for reagent loading and/or may be configured to condition filled cartridges prior to processing. This system may comprise a housing, such as a temperature-controlled chamber sized to receive an entire cartridge therein. The housing may comprise one or more integrated thermal elements configured to interface with and regulate a temperature of, in some variations, a blank cartridge (e.g., of the housing thereof). The conditioning system 188 may be positioned to receive cartridges directly from the receiving bay 182 via the RTS 184. The conditioning system 188 may regulate cartridge temperature to prevent, for example, thermal shock as a result of subsequent reagent handling, which could compromise the stability or performance of the cartridge during processing. In some variations, the conditioning system 188 may comprise one or more temperature control elements configured to maintain preset (e.g., by the control system 140) thermal conditions based on the reagents to be loaded into the given cartridge. The physical structure of the conditioning system housing may comprise thermally isolated mounting points for cartridges and may incorporate sensors that monitor cartridge temperature throughout the conditioning process.

Additionally, or alternatively, the conditioning system 188 may be configured to interface with and regulate a temperature of a filled cartridge that has been loaded with processing materials. That is, prior to completion of the preparation process, each filled cartridge may undergo final processing at the conditioning system 188. To achieve this, the conditioning system 188 may comprise a degasser configured to remove air from the cartridge and prevent bubble formation therein during subsequent processing stages. The conditioning system 188 may comprise temperature cycling capabilities, via the one or more thermal elements, to activate specific reagents if required by the workflow. In some variations, the conditioning system may further comprise a verification system configured for tomographic inspection of the loaded reagents and their distribution within the cartridge. This system may thus verify fluid flow paths through all cartridge components. In some variations, the verification system may be configured to execute one or more test procedures for fluidic channels and valves (e.g., of the FTS 162) and may confirm proper functionality of one or more of the cartridge components. In some variations, the conditioning system 188 may initialize internal control elements within the cartridge, preparing them for interaction with downstream instruments (e.g., processing and finishing instruments).

Moreover, in some variations, prior to dispensing, reagents may pass through the conditioning system 188 to bring them to an optimal temperature for subsequent handling and reactivity.

### Analytical System

The analytical system 189 may receive cartridges from the sealing system 187 via the RTS 184 and may be positioned near the final stages of the preparation workflow. The analytical system 189 may comprise a housing that supports one or more analytical tools. This system may be configured to coordinate with other verification systems of the preparation instrument 180 (described above) to analyze results throughout the cartridge preparation process. In some variations, the analytical system 189 may comprise an imaging tool, which may be mounted within the housing and may be configured to identify physical (e.g., geometric) or functional abnormalities of the cartridge or reagents therein. In some variations, such as for critical reagents, the analytical system 189 may comprise on or more scales to verify a weight of a reagent (e.g., with an accuracy of ±0.1 mg) and/or one or more spectrophotometric tools to confirm a concentration and/or purity of a reagent.

Moreover, in some variations, the analytical system 189 may include a labeling tool configured to generate a unique identifier (e.g., linked to patient and/or batch data) for the cartridge. As an example, a barcode printer or RFID tag applicator may be mounted within the analytical system 189 to affix these identifiers to each prepared cartridge, enabling digital verification and facilitating tracking throughout subsequent processes.

The analytical system 189 may generate stored reagent identity for materials with characteristic absorption profiles. This may comprise digital process records generated throughout the preparation sequence. In some variations, the records may comprise, for example, images at critical stages (e.g., after one or more steps of the preparation workflow are complete) to provide visual documentation of proper execution. In some variations, the records may comprise all process parameters, verification results, and exception notifications that occurred during preparation. The verification data and records may be transmitted in real-time to the control system 140, creating a comprehensive record of the preparation process in compliance with regulatory requirements.

The RTS 184 may transfer a prepared cartridge back to the receiving bay 182, which may be configured to maintain preset environmental conditions (E.g., temperature, light, etc.) until the cartridge is transferred to the next processing stage. The controller 140 may notify one or more components within the system 100, such as the RTS 104 and/or the processing instrument 120, that the given cartridge is prepared for processing, streamlining coordination.

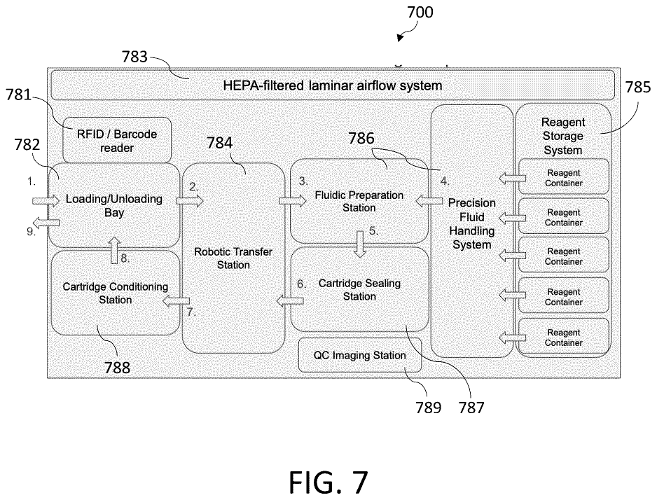

Referring to FIG. 7, an exemplary layout of a preparation instrument 700 is illustrated. The preparation instrument 700 may comprise a housing 781 that encloses an interior volume where various processing systems are arranged in an optimized workflow configuration. An upper portion of the housing 781 may comprise a HEPA-filtered laminar airflow system 783, which may be part of environmental management system of the instrument 700 (which may regulate, e.g., temperature, humidity, air pressure, and/or the like within the interior volume). The HEPA-filtered laminar airflow system 783 may extend across the top of the interior space, providing controlled environmental conditions throughout the workspace.

The preparation instrument 700 may comprise a receiving bay 782 positioned at a first side of the housing 781. An RFID/barcode reader 781 may be mounted adjacent to the receiving bay 782 for cartridge authentication and tracking. A cartridge conditioning system 785 may be positioned adjacent to the receiving bay 782, allowing for efficient transfer of newly received cartridges. Additionally, a robotic transfer system 788 may be centrally located within the housing 781, providing access to all processing stations through automated movements.

A fluidic preparation station and accompanying fluid handling system, which together may be considered a material handling system 786, may be positioned at a central location within the housing 781. The fluidic preparation station may be configured to temporarily store a blank cartridge within its housing. The fluid handling system may be positioned between the fluidic preparation station and a reagent storage station 787, which may be provided along a second side of the housing 781 opposite the first side (comprising the receiving bay 782). The reagent storage station 785 may comprise a plurality of reagent containers, which may be arranged in an array comprising a plurality of rows and/or a plurality of columns of containers to optimize space utilization while maintaining appropriate temperature conditions for each reagent type. The fluid handling system may be configured to transfer reagents from the reagent storage station 785 to one or more fluid compartments within the cartridge housed in the fluidic preparation station.

Further, the sealing station 787 may be positioned adjacent to the materials handling system 786 to facilitate receiving cartridges from the fluidic preparation station 786 via the robotic transfer system 784. Adjacent to the sealing station 788, a quality control imaging system 789, which may be part of a greater analytical system configured for additional analytical tests, may be positioned adjacent to the sealing station 787 perform final verification of prepared cartridges before they are returned to the receiving bay 782 for transfer to subsequent processing stages.

The arrangement of components within the preparation instrument 700 may be configured to optimize workflow efficiency, with numbered pathways (1-9) indicating the typical movement sequence of cartridges through the system. This organization may minimize transfer distances while maintaining appropriate separation between different processing stages to prevent cross-contamination. While FIG. 7 illustrates one possible arrangement of components, the components described with FIG. 1C above may be configured in various alternative layouts to accommodate specific facility requirements or manufacturing volumes, while maintaining the functional relationships between the various subsystems.

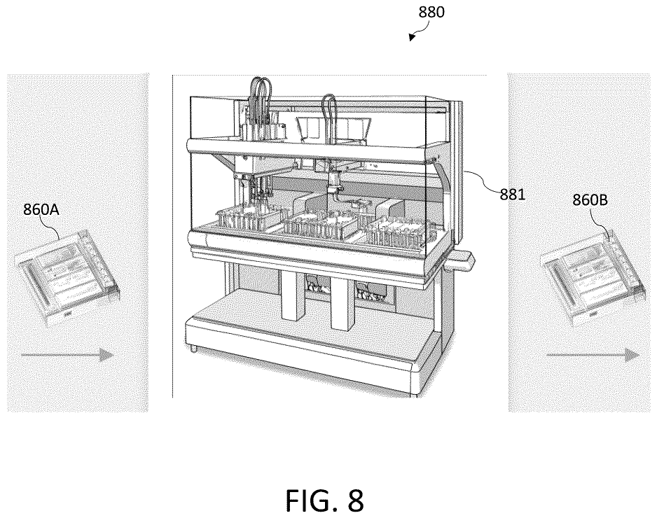

Finally, FIG. 8 depicts a pre-processing portion of an automated bioprocessing workflow. As shown, a blank version of the cartridge 860A may be loaded into a preparation instrument 880, which may fill/prepare/preprocess the cartridge 860A entirely within its housing 881. After the preprocessing workflow is complete, the preparation instrument 880 may output a filled/prepared version of the cartridge 860B.

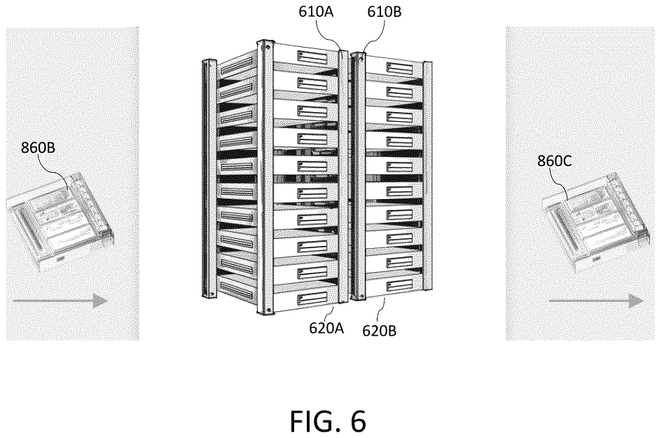

Once filled and prepared, the cartridge 860B can then be transferred to the one or more processing instruments, as shown in FIG. 6. Each of the one or more processing instruments within one or more support structures, such as processing instruments 620A/620B held by support units 610A/610B, respectively, may be configured to process nucleic acid material via a filled, pre-processed cartridge 860B. The cartridge 860B may be filled and pre-processed by a preparation instrument configured to load the cartridge with all reagents necessary for therapeutic manufacturing. After some or all of the processing workflow has been completed within each instrument, the instrument may eject a fully processed cartridge 860C. The cartridge 860C may be transported to a post-processing finishing instrument to ready a nucleic acid product therein for shipping and/or immediate clinical use. More details regarding the one or more processing instruments are described in Section III below.

### III. Processing Instrument

The processing instrument may define a second instrument class that is configured to manufacture a nucleic acid material via a preprocessed and filled cartridge. The processing instrument may receive the cartridge from the preparation instrument and perform one or more nucleic acid processing steps (e.g., amplification, transcription, digestion, capping & tailing, purification, and/or concentration) on the material held therein. In some variations, the processing instrument may execute all of these steps in a single continuous workflow, with instructions provided by a system-wide control system that also oversees any other instruments operating in parallel. Each instrument may be individually controlled by a processing-instrument-specific controller that automatically executes and, if necessary, adjusts the workflow based on data received from the instrument itself. In some variations, the system may comprise a plurality of such processing instruments, each working simultaneously on a different cartridge.

As shown in FIG. 1E, the processing instrument 120 may include a housing 121 configured to define an interior space, a receiving bay 122 within the interior space, an opening 123 for the receiving bay 122, a vent 124, a fluid transfer system 125, a temperature control system 126, an analytical system 127, and a plurality of actuating modules (e.g., an enzyme reaction actuating module 130, a purification actuating module 132, and a particle formation actuating module 134). The housing 121 may have a generally rectangular cross-section formed by a plurality of sidewalls. The housing 121 may be constructed of rigid or semi-rigid material, such as one or more metals, polymers, or composites, to maintain its shape. In some variations, the cross-sectional shape of the housing 121 may match that of the cartridge's housing to facilitate stable placement within the receiving bay 122. The housing 121 may have a height of about 2 in to about 30 in, such as about 3 to about 20 in, about 4 in to about 15 in, about 5 in to about 10 in, about 6 in to about 9 in, or about 7 in to about 8 in, etc. In some variations, the processing instrument 120 may be sized to fit within a support unit in a server rack-type arrangement (for example, about 3 U to 8 U, or specifically about 4 U in vertical dimension, where 1 U equals about 1.75 inches or 44.45 mm, to fit standard 19-inch server racks). Other configurations or custom housings may also be employed.

The receiving bay 122 may represent the sole functional zone within the interior space, helping to minimize an overall size of the housing 121. The receiving bay 122 may be a “cartridge nest” with internal dimensions that are about equal to or greater than corresponding external dimensions of the cartridge housing. Generally, a volume of the receiving bay 122 may be about equal to a volume of the cartridge housing to less than about 1.5× or 2× the volume of the cartridge housing. In some variations, the housing 121 may include one or more vents 124 in each of one or more sidewalls to facilitate air exchange or pressure equalization in portions of the instrument not in fluid communication with the cartridge. A filter or membrane could be positioned in or around the vent to maintain controlled interior conditions. The receiving bay 122 itself may include a cover, such as a slidable or rotatable door, configured to transition between an open configuration (permitting cartridge insertion or removal) and a closed configuration (securing the cartridge for sterile, controlled processing). The cover may be automatically and/or manually actuatable (e.g., via a robot and/or an operator). In some variations, once the cover is closed, it may remain in that position throughout the workflow to preserve environmental conditions. In some variations, the receiving bay 122 may comprise a sensor adjacent to the opening 123 that is configured to detect and verify a cartridge type and/or batch identification via a unique label upon insertion. For example, the sensor may comprise an RFID or barcode scanner that may transmit cartridge/patient/batch identification information from the cartridge's identifying label to the control system for verification. This verification may also ensure that the correct processing workflow is selected for each cartridge.

In some variations, the housing 121 may comprise one or more system inputs, such as one or more fluid (e.g., gas) inputs and one or more power inputs that are configured to couple with common gas and power lines of the system.

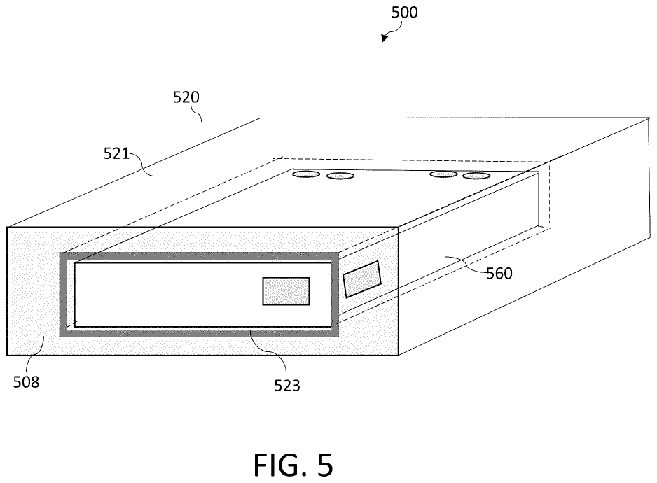

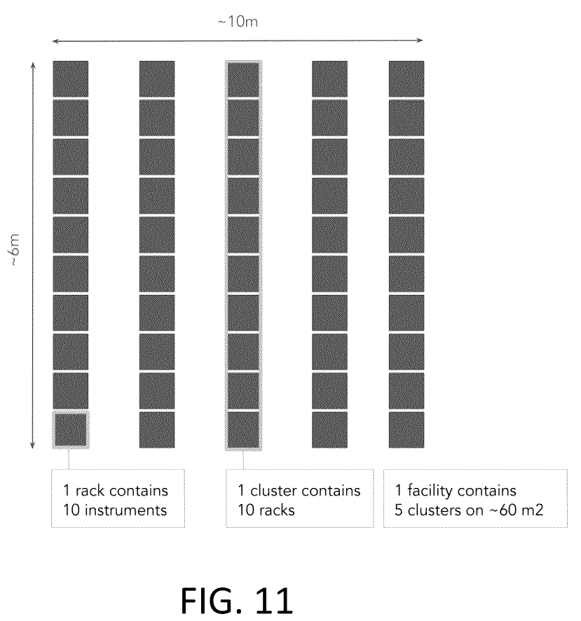

In some variations, the housing 121 may be configured to be arranged in a particular orientation relative to housings of additional processing instruments within the system 100. For example, the housing 121 may be stackable within and/or mountable onto a support unit comprising at least one additional processing instrument. All of the processing instruments may be arranged in a fixed configuration that is defined by the structure support unit, such as a column or row. The processing instrument 120 may be releasably coupled to the support unit to allow for replacements and maintenance operations. In some variations, the housing 121 may comprise one or more engagement features (e.g., mounts) for releasably attaching to the support unit. For example, the housing 121 may comprise two to four engagement features extending therefrom. Each engagement feature may be couplable to the support unit via one or more mechanical attachment mechanisms. For example, each engagement feature may be screwed into a portion of the support unit. As another example, each engagement feature may comprise a male or female engagement feature configured to couple with a corresponding female or male engagement feature on the support unit. The processing instrument configuration within a support unit will be discussed in further detail with respect to, for example, FIG. 5 and FIG. 11.

The processing instrument 120 may execute a workflow autonomously once a cartridge is loaded into the receiving bay 122. The workflow may be executing via the processing instrument's actuating modules, which may be configured to engage/interface with corresponding modules on the cartridge. The actuating modules (e.g., enzyme reaction, purification, and particle formation actuating modules) may each drive specific processing steps by interacting mechanically, magnetically, electronically, and/or thermally engaging/interfacing with corresponding cartridge modules. Additionally, like the cartridge, the processing instrument 120 may include several operating systems designed to monitor and/or control process parameters throughout the bioprocessing workflow. The operating systems may also be configured to engage/interface with corresponding cartridge systems to achieve an overall function (e.g., fluid transfer, temperature control, or product/process parameter monitoring). In some variations, a processing instrument actuating module and at least one instrument operating system may share one or more of the same components (e.g., fluid conduits, motors, magnets, stirrers, sensors, thermal elements, engagement features, and/or the like). The operating systems may generally include a fluid transfer system (FTS) 125, a temperature control system (TCS) 126, and an analytical system 127, each of which is described in detail below.

### Fluid Transfer System (FTS)

The FTS 125 may manage liquid routing within the cartridge. For example, the FTS 125 may comprise one or more pump actuators and/or pressurized air lines that connect to the fluidic pathways of the cartridge. In some variations, the cartridge may lack an on-board pump for controlling fluid transfer therein. To make up for this, in some variations, FTS 125 may be configured to move fluid through the cartridge using a pressure-driven approach rather than a dedicated pump on the cartridge itself. More specifically, a controlled flow of compressed air (or another inert gas) may be introduced into the cartridge's fluid conduits via one or more ports that align with corresponding connections of the FTS 125. By applying positive air pressure to a reagent or buffer compartment, the processing instrument 120 may push the fluid downstream through the sequence of fluid compartments. In this manner, the fluid may be guided from module to module in a controlled way, as directed by on-board valves or flow regulators within the cartridge.

This pump-less design may simplify the FTS 125 and the cartridge, reduce mechanical complexity, and improve cleanliness, since only controlled bursts of pressurized air are required to shuttle fluids between each stage of the workflow. Flow rates and pressures may be adjusted under the control system's direction, for instance through valves or regulators integrated with the processing instrument 120 or the cartridge.

### Temperature Control System (TCS)

The TCS 126 may regulate thermal conditions around one or more cartridge modules and/or stations. To do so, the TCS 126 may include one or more thermal elements distributed throughout the receiving bay 122. In some variations, the one or more thermal elements may be provided on a baseplate defining a lower perimeter of the receiving bay 122. Additionally, or alternatively, one or more of the thermal elements may be provided on an opposing top plate on an upper perimeter of the receiving bay 122. These elements (e.g., Peltier devices) may be configured to heat or cool localized portions of the cartridge that require specific temperatures. For example, one or more thermal elements may be positioned to align with cartridge modules configured for enzymatic reactions (e.g., the amplification module, transcription module, digestion module, and/or capping & tailing module). As another example, one or more thermal elements of the TCS 126 may be positioned such that they each align with one or more fluid compartments of the cartridge's storage station. This way, the TCS 126 may maintain optimal temperatures for reagents and/or product throughout the processing. In some variations, each thermal element may be configured for bidirectional temperature control to enable distinct zones of the cartridge and maintain different target temperatures. In some variations, the TCS 126 may comprise partitions constructed of thermally-isolating material to further separate the distinct temperature zones.

### Analytical System

The analytical system 127 may collect real-time data on process and/or product parameters throughout some or all of a processing workflow. For example, this system may be configured to collect real-time data from the cartridge, such as temperature, pressure, flow rate, pH, conductivity, optical density, UV absorbance, or particle size. To do so, the analytical system 127 may comprise one or more sensors, such as one or more optical sensors (e.g. cameras, bubble sensors), spectroscopic sensors, conductivity probes, particle analyzers, and/or the like. Each sensor of the analytical system may be communicably coupled with a controller to enable the control system to analyze the sensor data and adjust process parameters accordingly, thereby enabling closed-loop control and continuous monitoring of product quality. In some variations, the analytical system 127 may be partially or fully integrated with the cartridge analytical system 169. That is, the analytical system 127 may comprise one or more of the same components as the cartridge analytical system 169.

In some variations, one or more sensors of the analytical system 127 may be part of a feedback loop whereby the control system 140 is configured to make adjustments based on calculated parameters. For example, in some variations, one or more of a pH, conductivity, temperature, pressure, and/or the like may be pre-set (e.g., by the controller 140) to be maintained within a particular range. If a sensor detects a parameter outside of the range, the control system 140 may be configured to (i) alert an operator, (ii) pause or cease functioning for a set period of time or indefinitely, and/or (iii) self-correct.

Referring now to the actuating modules on the processing instrument 120, these actuating modules may include an enzyme reaction actuating module (configured to regulate one or more of amplification, transcription, digestion, and a capping & tailing), a purification actuating module configured to regulate purification, and a particle formation actuating module configured to regulate LPN particle formation. The housing 121 and its internal components may be arranged so that these actuating modules remain in a fixed or reconfigurable arrangement. In some variations, various component subassemblies (e.g., fluid lines, motors, magnets, stirrers, sensors, thermal elements) may be interspersed yet grouped functionally by the control system as a single actuating module. The actuating modules are described in more detail below.

### Enzyme Reaction Actuating Module (ERAM)

The ERAM 130 may actuate one or more of the transcription, digestion, and capping & tailing modules of the cartridge. In doing so, the ERAM 130 may ensure homogenous reaction conditions within these modules (e.g., within a mixing chamber thereof). In some variations, one or more modules of the cartridge may be actuated simultaneously by the ERAM 130. Accordingly, the ERAM 130 may drive one or more of the transcription, digestion, and capping & tailing steps of mRNA manufacturing. To do so, the ERAM 130 may interface with one or more of the aforementioned modules via a magnetic coupling. In particular, a magnetic stirrer or mixing paddle within a fluid compartment of the cartridge may be drawn to the magnet of the ERAM 130. This magnet may be rotatable, and may have a variable operational speed of about 0 RPM to about 500 RPM, such as about 25 RPM to about 400 RPM, about 50 RPM to about 350 RPM, about 75 RPM to about 300 RPM, about 100 RPM to about 250 RPM, or about 150 RPM to about 200 RPM (including all ranges and subranges therein). The speed of the rotatable magnet of the ERAM 130 may be controlled by the control system. In some variations, the rotatable magnet, via instructions from the control system, may be configured to apply one or more programmable mixing patterns within the transcription, digestion, and/or capping & tailing modules.

### Purification Actuating Module (PAM)

The PAM 132 may include one or more sub-actuating-modules for actuating corresponding submodules of the purification module of the cartridge. The sub-actuating-modules may include one or more of a tangential flow filtration actuating module (TFFAM), a magnetic selection actuating module (MSAM), and a chromatography actuating module (CAM).

The TFFAM can be configured to automatically control a pressure differential transversely across the membrane of the TFFM, and to regulate a flow rate of fluid longitudinally across the membrane of the TFFM. The TFFAM may achieve automated pressure control via a pressure regulator coupled to a compartment of the TFFM. Additionally, the TFFAM may be configured to control the diafiltration cycle during TFF by utilizing the FTS 125 to move fluid through the TFFM to control buffer exchange.

The MSAM may comprise one or more permanent magnets mounted on a mechanism (e.g., a swivel) that is configured to engage a flowcell on the cartridge. Accordingly, the MSAM may enable magnetic separation used to purify an mRNA intermediate product. Additionally, the MSAM may include one or more sensors, such as bubble sensors and/or cameras, to monitor one or both of a fluid level and a flow rate within the flowcell. Further, the MSAM may comprise one or more optical sensors (e.g., bubble sensors and/or cameras) to detect a fluid level within a fluid compartment (e.g., reaction chamber) of the cartridge magnetic selection module. The one or more optical sensors may transmit data to the control system 140 so that, when the fluid level and/or flow rate is about equal to or greater than a threshold, indicating that the flowcell may overflow, the magnetic separation process may be modified to relieve and/or prevent such overflow. Thus, the MSAM may advantageously help prevent device and system damage by monitoring fluid transfer within the cartridge 160.

Like the TFFM, the CAM may utilize the FTS 125 to move deliver buffer for the cartridge chromatography module. Additionally, the CAM may operate one or more columns of the chromatography using valves. Further, the CAM may include one or more pressure sensors configured to detect pressure within the one or more columns and transmit the data to the control system 140. When the measured pressure is about equal to or greater than a threshold (determined by the control system 140), the chromatography process may be modified to relieve the pressure therein. Thus, the CAM may help prevent device and system damage by monitoring pressure within the cartridge 160. Additionally, the CAM may utilize one or more thermal elements of the TCS 126 for controlling a temperature of the cartridge chromatography module.

### Particle Formation Actuating Module (PFAM)

The PFAM 134 may employ the FTS 125, thermal elements of the TCS 126, as well as automated cleaning and flushing procedures (via, e.g., the control system 140) to maintain optimal conditions for the formation of lipid nanoparticles encapsulating mRNA within the particle formation module of the cartridge 160.

In summary, the processing instrument 120 may comprise the housing 121 (optionally fitting a standard rack form factor) enclosing the receiving bay 122, which may comprise the opening 123. In some variations, the housing 121 may further comprise a cover configured to close over the receiving bay 122 for sealed processing. Additionally, or alternatively, in some variations, the housing 121 may further comprise one or more vents for passive environmental control of the interior space. The processing instrument 120 may integrate a plurality of actuating modules and a plurality of operating systems that interact with a correspondingly equipped cartridge to enable or monitor the processing workflow. By housing these functionalities in a fixed or reconfigurable layout, the processing instrument 120 may be adapted for various scales or protocols of RNA manufacturing. When placed in parallel within a support unit (e.g., a mountable rack), a plurality of such processing instruments may simultaneously process a plurality of nucleic acid materials (e.g., each for a different patient) under a single control framework of the system 100.

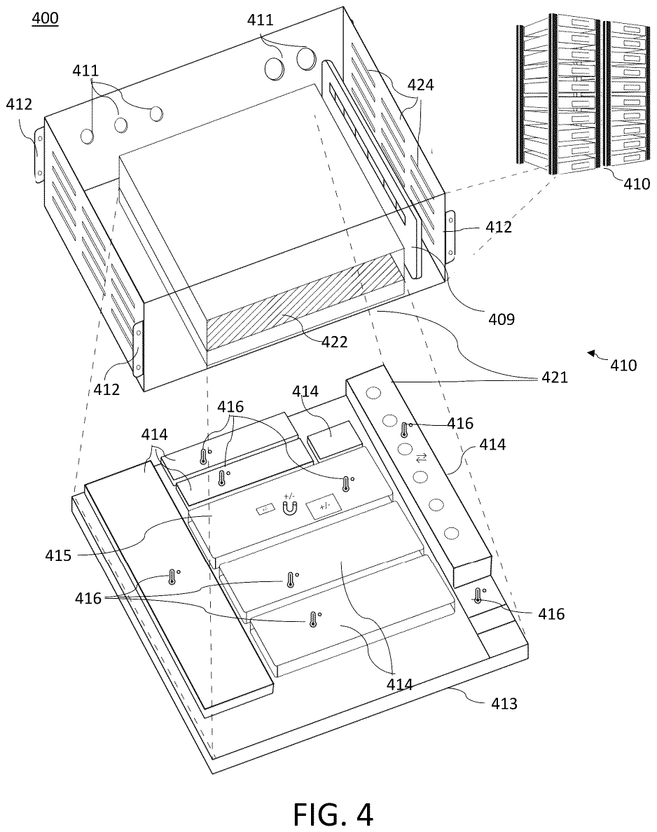

FIG. 4 depicts a partially exploded view of a portion of an automated system for bioprocessing 400. Specifically, the system 400 may comprise a plurality of processing instruments organized within one or more support units 410 (e.g., at least two support units, as shown). This may optimize space utilization within a larger system enclosure (not shown) housing the support units 410. A first processing instrument 420 is shown in a partially exploded view. The processing instrument 420 may be configured to receive a cartridge like the cartridge 360 shown in FIG. 3. The processing instrument 420 is shown with a lid/upper portion of its housing 421 being transparent to depict a receiving bay 422 therein. The receiving bay 422 may comprise an opening 423 in a front sidewall (also shown transparently) of the housing 421 that is sized and shaped to receive a cartridge (not shown). The interior space within the housing 421 may also house a sensor interface 409.

The housing 421 may further comprise vents 424 within one or both lateral sidewalls thereof, inputs (e.g., gas and power inputs) 411 within a back sidewall of the housing 421, and engagement features (e.g., mounts) 412 extending from corners (e.g., all four corners) of the housing 121. Further, the housing 412 may be partially defined by the base 413, which may comprise a substantially planar frame affixed thereto one or more components of the actuating modules/systems of the processing instrument 420. For example, the base 413 may comprise, for example, one or more thermal elements 414 distributed along its surface. The base 413 may comprise a magnet 415 configured to actuate the cartridge purification (e.g., debeading) module. The base 413 may additionally comprise one or more connectors 416, which may comprise alignment features, fluid (e.g., gas) inputs, and/or power inputs.

FIG. 5 depicts a portion of an automated system for bioprocessing 500. In particular, a perspective view of cartridge 560 is shown within a processing instrument 520, with a lid/upper portion 509 of the processing instrument 520 shown transparently to depict the cartridge 560 therein. The processing instrument 520 may comprise an opening 523 within a first, front face 508 of the processing instrument 520. The cartridge 560 may be configured to be inserted through the opening 523 until it is fully situated within a receiving bay (not shown) of the processing instrument 520. In some variations, the opening 523 may be coupled to a cover (not shown) that is configured to be moved to a closed position (in which the opening is covered by the cover) when the cartridge 560 is within the receiving bay, resulting in a closed processing environment for nucleic acid material contained within the cartridge 560 that is enclosed by the processing instrument 520.

FIG. 6 depicts a processing portion of an automated bioprocessing workflow. As shown, each of one or more processing instruments within one or more support structures, each of the plurality of processing instruments 620A/620B held by support units 610A/610B, respectively, may be configured to process nucleic acid material via a filled, pre-processed cartridge 860B. The cartridge 860B may be filled and pre-processed by a preparation instrument configured to load the cartridge with all reagents necessary for therapeutic manufacturing. After some or all of the processing workflow has been completed within each instrument, the instrument may eject a fully processed cartridge 860C. The cartridge 860C may be ready transported to a post-processing finishing instrument to ready a nucleic acid product therein for shipping and/or immediate clinical use.

### IV. Finishing Instrument

The finishing instrument may perform the final step in a personalized therapeutics manufacturing workflow by transforming processed cartridges into patient-ready therapeutic products. The finishing instrument may be configured to extract a nucleic acid product contained within the cartridge to execute quality control and fill/finish steps on the nucleic acid product. The finishing instrument may combine precision extraction, analytical testing, and sterile fill/finish operations within a compact, self-contained enclosure providing all necessary functionality for consistent, accurate, and compliant therapeutic product manufacturing. In some variations, the finishing instrument may be configured to finish (“process”) a single cartridge at a time. Alternatively, the finishing instrument may be configured to process a plurality of cartridges simultaneously. Thus, the finishing instrument may ensure that the therapeutics produced meet consistency and quality criteria (e.g., regulatory requirements) for personalized therapies.

The system may comprise one or more finishing instruments, such as a plurality thereof. In some variations, one or more finishing instruments 190 may be configured to finish (“process”) a single cartridge at a time. Alternatively, one or more finishing instruments may be configured to process a plurality of cartridges in parallel.

A block diagram of an exemplary variation of the finishing instrument 190 is provided in FIG. 1E. The finishing instrument may generally comprise a housing 191, a receiving bay 192, an environmental management system (EMS) 193, a robotic transfer system (RTS) 194, a sterilization system 195, a materials handling system (MHS) 196, an extraction system 197, an analytics system 198, and a filling system 199.

The housing 191 may be configured to maintain a sterile environment therein, providing a controlled space critical for maintaining product sterility throughout all operations. The housing 191 may include sidewalls that define an interior space and may fully or at least partially enclose the components of the finishing instrument 190. The housing 191 may comprise a filter system, such as a HEPA-filtered laminar airflow system, to maintain sterile conditions within the interior space. The filter system may extend across at least a portion of an upper portion of the interior space, directing filtered air downward to maintain positive pressure and prevent contamination. In some variations, the filter system may be configured to meet ISO8 or better conditions (e.g., ISO 5/Grade A requirements) through HEPA-filtered unidirectional airflow that covers the processing areas. This airflow system may continuously sweep the processing areas to eliminate potential contaminants, minimize dead zones, and create a contamination-free zone for critical operations.

The environmental management system (EMS) 193 may be integrated with the housing 191 and may be configured to provide environmental control within the interior space, ensuring optimal conditions for all finishing operations. For example, the EMS 193 may maintain positive pressure differential relative to the surrounding environment, employ an ordered flow pattern, and/or regulate environmental additional conditions (e.g., temperature and humidity) within specified ranges. In some variations, the EMS 193 may include temperature and humidity sensors distributed throughout the interior space, which may be coupled to the control system 140 to enable real-time adjustment of environmental parameters. This comprehensive environmental monitoring and control capability may help maintain product quality throughout the finishing process. In some variations, throughout some or all of the finishing operations, the EMS 193 may be configured to maintain an aseptic chain with little to no risk of the product or cartridge opening to an environment less controlled than ISO8 or better (e.g., ISO5/Grade A).

The receiving bay 192 may be positioned within a sidewall of the housing 191, serving as an interface between the processing and finishing stages of the manufacturing workflow. The receiving bay 192 may function as a controlled-access port that may be configured to receive a processed cartridge (e.g., via the RTS 104) under controlled, sterile conditions. An RFID or barcode reader may be positioned adjacent to the receiving bay 192 to confirm cartridge identity, authenticate batch information, and retrieve relevant production data associated with the product. This verification step ensures that the correct cartridge is processed and maintains the chain of identity throughout the manufacturing process.

An interior space of the finishing instrument 190 may be compartmentalized into several discrete operational zones. In some variations, the interior space of the finishing instrument 190 may be structured to provide a defined workflow path that minimizes cross-contamination risks while optimizing process efficiency. Interior partitions or barriers may separate different functional areas while allowing for robotic or automated transfer of materials between zones. This compartmentalization may enhance both operational efficiency and contamination control throughout the finishing workflow.

The robotic transfer system (RTS) 194 may be configured to transport the cartridge to various systems within the finishing instrument housing 191, providing automated cartridge transport throughout the finishing process. The RTS 194 may be mounted on a frame that extends through central portions of the interior space, providing access to each operational station. In some variations, the RTS 194 may utilize end effectors designed to securely handle cartridges and sample containers. These end effectors, which may be grippers, may be attached to one or more robots, such as one or more articulated arms or gantry systems capable of multi-axis movement throughout the interior workspace. In some variations, RTS 194 may utilize a track (e.g., a linear guiderail) to move throughout the housing 191. This automated transport system may minimize contamination risks associated with convention manual touchpoints throughout nucleic acid product finishing.

### Sterilization System

The sterilization system 195 may be integrated with the extraction system 197 and may include a decontamination system, such as a vaporized hydrogen peroxide (VHP) system or ultraviolet (UV) decontamination system, configured to sanitize one or more connections (e.g., fluid ports) on the cartridge. This critical system ensures sterility at all connection points, maintaining product integrity. The sterilization system may include, for example, one or more spray nozzles, vapor generators, or UV light sources mounted on adjustable arms that may be positioned to target specific cartridge surfaces. The sterilization system 195 may enable the extraction system 197 to establish a sterile connection to the interior of the cartridge while maintaining the controlled environment.

### Extraction System

The extraction system 197 may be configured to open a closed cartridge such that a nucleic acid product stored therein (e.g., in its storage station, such as within a product compartment thereof) may be extracted therefrom. The extraction system 197 may be positioned to receive cartridges via the RTS 194, continuing the automated workflow. The extraction system 197 may comprise a chamber configured to support the cartridge throughout cartridge unsealing and product extraction operations. The chamber may be structured with reinforced mounting points to securely position the cartridge during the extraction process. In some variations, the chamber may comprise one or more controlled environmental conditions. For example, the chamber may maintain controlled environmental conditions while the system performs integrity verification of the cartridge, confirming that no damage has occurred during transit from the previous instrument.

The extraction system 197 may further comprise an extractor configured to penetrate and/or mate with an access port of the cartridge. This connection may thus unseal the cartridge and enable access to at least a portion of its interior, such as to one or more fluid compartments of its storage system. In some variations, the extractor may comprise a precision needle, specialized tubing, or a docking interface with multiple verification points designed to confirm proper seating and alignment. The connection between cartridge and instrument may establish a sterile fluid-tight path enabling closed-system transfer technology that prevents microbial ingress during the critical extraction process. The extractor may be coupled to the materials handling system 196 that controls the fluid extraction process.

### Materials Handling System (MHS)

The MHS 196 may be positioned adjacent to the extraction system 197 and may comprise a series of precision pumps, valves, sensors, and fluid pathways that form the core fluid management infrastructure of the finishing instrument 190. These components may be constructed of single-use components that undergo pre-use integrity verification to reduce or even completely eliminate cross-contamination risk between patient-specific products. The MHS 196 may comprise a plurality of pump assemblies, each with independently controlled channels capable of small volumetric transfers. For example, the MHS 196 may be configured to handle volumes ranging from about 0.01 μL to about 50 mL (e.g., about 1 μL to about 20 mL). This small-batch optimized configuration may help minimize product loss while ensuring sufficient material for accurate testing. In some variations, the MHS 196 may be mounted on a structural framework within the housing 191 that allows for modular replacement of components while maintaining system integrity.

Further, the MHS 196 may be configured with a plurality of fluid channels to enable concurrent handling of product samples, reagents, and quality control materials. The channels may be arranged in a compact configuration with minimal dead volume to optimize recovery of the nucleic acid product. The MHS 196 may interconnect the various stations of the finishing instrument 190 through a network of sterile tubing, transfer pipettes, and automated connectors. Following extraction from the cartridge, the nucleic acid product may be transferred to sample containers via a pipettor (e.g., a micropipettor) integrated within the MHS 196. This pipettor may comprise multiple channels with independently controlled disposable tips capable of handling volumes on the order of, for example, microliters to milliliters. The pipettor may be mounted on a movable arm that may access both the extraction point and various downstream processing stations.

### Analytical System

Once the extracted product is ready for quality assessment, the analytical system 198 may be utilized. This system may be positioned within the interior space to receive samples from the MHS 196 via the RTS 194, maintaining the automated workflow sequence. The analytical system 198 may comprise a plurality of analytical tools, each capable of performing specific analytical tests on an extracted sample to ensure product quality and compliance with specifications. In some variations, the analytical tools may be arranged in a compact array configuration. Additionally, or alternatively, the analytical tools may be mounted on a common structural platform with standardized connections for power, data, and fluid transfer. For example, the analytical system 198 may comprise equipment such as spectrophotometers, chromatography vials, or PCR units, and each may be secured within dedicated mounting brackets or frames to ensure stability during operation. In some variations, one or more analytical tools may be arranged to allow robotic access for sample placement and retrieval, with sufficient clearance between units to facilitate maintenance. In some variations, the analytical system 198 may comprise a visual-based verification system that provides independent confirmation of fluid handling accuracy, particularly critical for patient-specific dosing.

To maintain product traceability throughout testing, each sample may be dispensed into a sample container, which may be specific to an individual analytical method. For example, liquid chromatography vials may be used to hold samples prior to analysis to prevent evaporation or contamination. Similarly, PCR tubes or plates may be used to store samples for PCR tests. In some variations, the analytical system 198 may apply a unique barcode to each container to establish traceability throughout the quality control process. These comprehensive analytical capabilities ensure that each product meets all required specifications before release. Following successful quality testing, the product may be transferred to the filling system 199.

### Filling System

The filling system 199 may be configured to prepare a final therapeutic product for delivery. Thus, the filling system may execute the final steps of the finishing and overall processing workflow. The filling system 199 may be positioned within a second (e.g., distal) portion of the interior space that is opposite a first (e.g., proximal) portion comprising the receiving bay 192. The filling system 199 may comprise one or more product containers, as well as a container preparation and sterilization system. The sterilization system may be the sterilization system 195, and may include one or more UV sterilization chambers, laminar flow hoods, and/or other decontamination systems. The filling system 199 may further comprise filling equipment with volumetric dispensing capabilities. The filling equipment may be mounted on vibration-isolated platforms to ensure accurate dosing of a product into a product container. In some variations, the product container may be a vial.

To ensure precise and sterile delivery of the final product, the filling system 199 may verify container sterilization status and position each container precisely during filling operations. The filling equipment may include high-precision dosing pumps capable of delivering volumes with volumetric accuracy verified by weight measurement, ensuring consistent dosing critical for therapeutic efficacy. The filling system 199 may comprise an integrated stopper or closure placement system (having, e.g., mechanical arms or pneumatic actuators) to seal containers according to product specifications.

To maintain product integrity throughout, the packaging process filling system 199 may perform automated stopper or closure placement within the aseptic environment, minimizing exposure risk. This may be carried out immediately after the filling operations. The automated placement may include crimping or sealing the closures relative to/with their product containers before the product is transferred away from the filling equipment. The filling system 199 may be configured to confirm a closure level or integrity through visual inspection, force verification, and/or leak testing depending on container type.

In some variations, the finishing instrument 190 may further comprise a temperature control system (TCS) positioned adjacent to the filling system 199. The TCS may be used for products requiring specialized storage conditions. As such, the TCS may comprise one or more cooling elements, temperature sensors, and structural supports designed to securely hold containers during the cooling process. The cooling elements may be arranged in a configuration that ensures uniform temperature distribution around the product containers. The temperature control system may further include insulated compartments with sealed access points to maintain cold chain integrity when transferring finished products. In some variations, the TCS may be configured to maintain a range of temperatures including about −200° C. to about 50° C. (e.g., about −196° C. to about 40° C.), accommodating diverse product storage requirements. In some variations, the TCS may comprise a cryopreservation system configured to implement controlled freezing of the products at rates from about 1° C./min to about 100° C./min. In some variations, a freezing rate for a product may be predetermined and optimized for a final formulation of the product. Upon reaching the target storage requirements, an integrated transfer system may move the frozen products to temporary storage containers or to representative containers to document the freezing profile. The system may include a temperature-controlled protocol which continuously monitors critical parameters and documents the complete freezing curve for quality review.

To facilitate product identification and traceability, in some variations, the finishing instrument 190 may additionally comprise a labeling system, which may include printing hardware, application mechanisms, and verification cameras mounted within the housing 191. The application mechanisms may include precision positioning components to ensure accurate label placement on various container types. The 1 labeling system may be positioned near the filling system 199 to minimize product handling. In some variations, the labeling system may be configured to provide on-demand label printing capabilities that generate patient-specific information at time of use, eliminating mix-up risks associated with pre-printed labels.

Further enhancing product security and identification, the label application verification may include a vision system that confirms proper label positioning and print quality before application. Patient-specific information may be applied following strict verification protocols that maintain the chain of identity from prescription to final container. Two-dimensional barcodes may incorporate order-checking algorithms, while tamper-evident features provide visual indication of container access after manufacturing.

For products requiring immediate distribution, the finishing instrument 190 may prepare shipping containers with temperature monitoring and control features appropriate for the specific product requirements. Throughout the packaging process, temperature sensors may be integrated with the product packaging to document environmental conditions during subsequent handling. A final reconciliation of product may be performed prior to release, completing the comprehensive finishing process.

Like the other instruments herein, the finishing instrument 190 may be coupled to the control system 140, ensuring integrated control and monitoring across the entire manufacturing process. In some variations, the finishing instrument 190 may comprise a control subsystem with integrated monitoring capabilities that is part of the control system 140. Via its controller, the finishing instrument 190 may be configured to maintain records of environmental conditions, process parameters, and quality control results through integrated sensors positioned throughout the housing 191. For example, sensors of the finishing instrument 190 may be mounted at critical monitoring points, connected to the control system via protected data pathways. In some variations, the finishing instrument 190 may be structured to enable connection with external data management systems, with communication ports or interfaces located on exterior surfaces of the housing 191. The control system may integrate real-time laboratory information management systems (e.g., LIMS), dramatically generating regulatory documentation and assurance with minimal touch points.

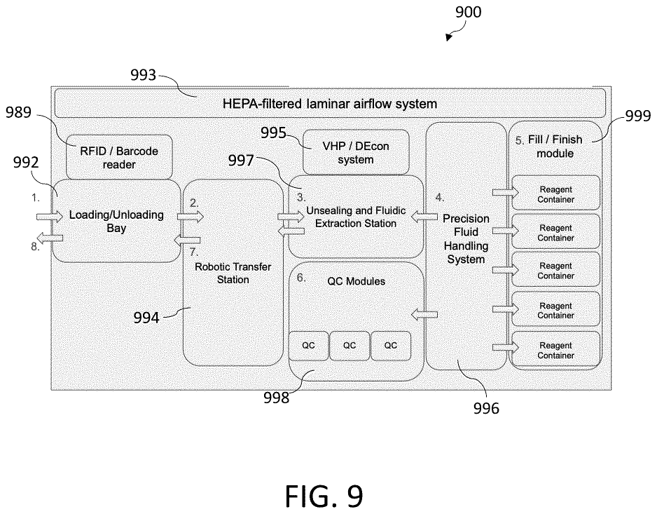

Referring to FIG. 9, an exemplary layout of a finishing instrument 900 is illustrated. The finishing instrument 900 may comprise a housing that encloses an interior volume where various processing systems are arranged in an optimized workflow configuration. An upper portion of the housing may comprise a HEPA-filtered laminar airflow system 993, which may be part of the environmental management system of the instrument 900 (which may regulate, e.g., temperature, humidity, air pressure, and/or the like within the interior volume).

The finishing instrument 900 may comprise a receiving bay 992 positioned at a first side of the housing. An RFID/barcode reader 989 may be mounted adjacent to the receiving bay 992 for cartridge authentication and tracking. A robotic transfer station 994 may be centrally located within the housing, providing access to all processing stations through automated movements.

The finishing instrument 900 may comprise an unsealing and fluidic extraction station 997, which may include a vaporized hydrogen peroxide (VHP) decontamination system 995. The finishing instrument 900 may further comprise a precision fluid handling system 996 positioned adjacent to the extraction station 997. A quality control (QC) module 998 comprising a plurality of analytical tools (e.g., configured for quality control operations) may be positioned near the precision fluid handling system 996. A fill & finish module 999 may be positioned at a second side of the housing opposite the first side, with product containers arranged therein for storing finalized nucleic acid products.

The arrangement of components within the finishing instrument 900 may be configured to optimize workflow efficiency, with numbered pathways (1-8) indicating the typical movement sequence of cartridges through the system. This organization may minimize transfer distances while maintaining appropriate separation between different processing stages to prevent cross-contamination.

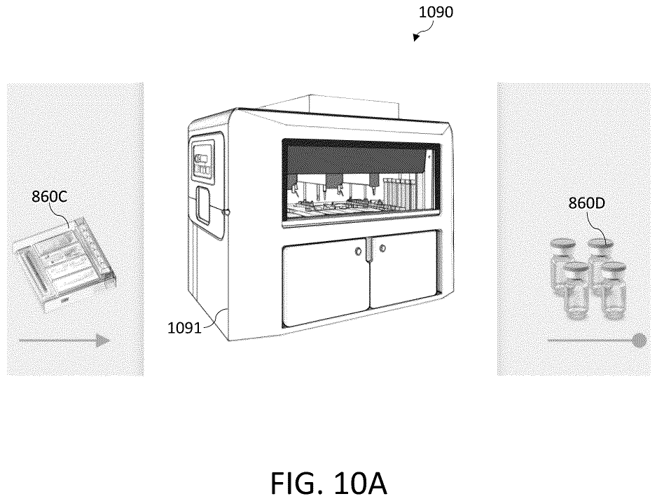

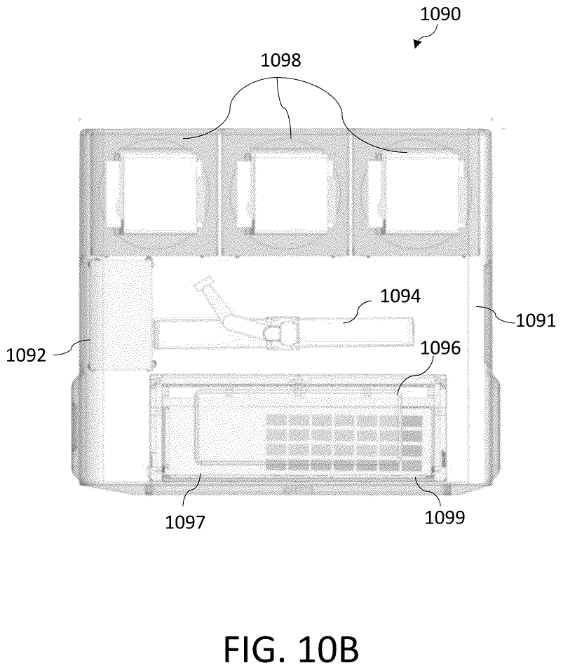

FIG. 10A depicts a post-processing portion of an automated bioprocessing workflow. As shown, a processed version of the cartridge 860C may be loaded into a finishing instrument 1090, which may extract and finish the nucleic acid product entirely within its housing 1091. After the finishing workflow is complete, the finishing instrument 1050 may output the final therapeutic product 860D in appropriate containers ready for clinical use.

Further, FIG. 10B depicts a top view of the finishing instrument 1090 utilized in the workflow of FIG. 10A. A top of its housing 1091 is provided transparently to show several components therein. As shown, the finishing instrument 1090 may comprise a receiving bay 1092, an RTS 1094 a materials handling system 1096, an extraction system 1097, an analytical system 1098, and a filling system 1099. In some variations, the analytical system 1098 may comprise a plurality of tools, such as three or at least three analytical tools. The analytical system 1098 may comprise, for example, one or more of each of a qPCR system, a flow-based particle analyzer, a microplate reader, and a nucleic acid fragment analyzer.

### V. Support Unit

The system 100 may include one or more support units 110 configured to hold instruments therein. In some variations, one support unit may be configured to support one or more of (e.g., a plurality of) a same instrument type—i.e., one or more preparation instruments, one or more processing instruments, or one or more finishing instruments—to maintain an organized system. For example, each of a plurality of support units 110 may be configured to hold at least two processing instruments 120 therein, such as at least four, at least six, at least eight, at least ten, or more than ten processing instruments therein. Additionally, a plurality of support units 110 may be arranged in a hierarchical format, such that each support unit is positioned within a cluster and each cluster includes a set of support units grouped in close proximity. By customizing both the dimensions of each support unit and the spatial relationships among the pluralities of support units and clusters, the system herein may achieve flexible, scalable, and space-efficient deployment. This architecture in turn may enable high-throughput processing of tens, dozens, hundreds, or potentially thousands of nucleic acid materials in parallel, each loaded into a separate processing instrument and cartridge

### Structure

The support unit 110 may comprise a housing that at least partially surrounds or encloses an interior volume of the support unit 110. In some variations, the housing may comprise a rack or a frame. The housing may comprise a rigid or semi-rigid material (e.g., a metallic or composite frame) designed to withstand the weight and operational requirements of multiple instruments. As an example, the housing may include one or more sidewalls (e.g., at least one sidewall) and/or one or more rails that border its interior volume. The one or more sidewalls or rails may be, for example, mounting sidewalls or rails configured to engage respective instruments. For example, at least one sidewall may comprise an engagement feature (or a plurality thereof, such as at least two engagement features) configured to couple with corresponding engagement feature(s) on a plurality of instruments. Additionally, or alternatively, at least one rail (e.g., at least two rails, such as a pair of opposing rails) may comprise engagement feature(s) configured to couple with corresponding engagement feature(s) on a plurality of instruments. In either case, the corresponding engagement features may comprise, for example, complementary male/female connectors, hook-and-slot arrangements, or adjustable clamp mechanisms that secure instruments in position while allowing for their removal when necessary.

In some variations, the housing may comprise one or more access panels that are movable between an open position and a closed position to provide selective access to the instruments contained within the support unit 110. These access panels may be hinged, sliding, or removable, and may cover part or all of the front, rear, sides, or top of the support unit. Moreover, the support unit 110 may optionally comprise one or more environmental control elements configured to maintain predetermined environmental conditions (e.g., temperature, humidity, or air filtration) within the interior volume of the support unit. Such environmental control elements may be disposed on interior surfaces (e.g., interior sidewall surfaces) of the housing and may include sensors that monitor environmental parameters, heating or cooling elements that regulate temperature, humidifiers or dehumidifiers, and air filtration systems that maintain appropriate particulate levels for bioprocessing operations.

In some variations, the support unit 110 may be configured to be mounted on a floor panel, hung from a ceiling grid, or attached to a sidewall of a larger enclosure (e.g., enclosure 101). Such mounting options may maximize available floor space and provide flexibility in facility design. Alternatively, the support unit 110 may be free-standing and simply positioned within the enclosure according to available space or workflow considerations. Further, in some variations, the support unit 110 may be movable, such as via one or more moveable elements (e.g., casters, wheels, glides, or adjustable feet) affixed to a bottom portion of the housing. This feature may facilitate repositioning of the support unit 110 within the enclosure 101, enabling dynamic reconfiguration of the system as processing requirements change over time. In alternative variations, the support unit may be configured for fixed installation, which may provide additional stability and reduce vibration during instrument operation.

The support unit 110 may comprise a same or substantially similar cross-sectional shape as the instruments it is configured to support (e.g., rectangular, square). A width of the support unit 110 may be about 5 in to about 100 in, such as about 10 in to about 50 in, about 15 in to about 25 in, about 17 in about 18 in, about 19 in, about 20 in, about 21 in, about 22 in, etc. A depth of the support unit 110 may be about 10 in to about 60 in, such as about 15 in to about 55 in, about 20 in to about 50 in, about 25 in to about 45 in, or about 30 in to about 40 in. A height of the support unit 110 may be about 15 in to about 95 in, such as about 30 in to about 90 in, about 50 in to about 85 in, about 60 in to about 80 in, about 65 in to about 75 in, about 68 in, about 69 in, about 70 in, about 71 in, about 72 in, about 73 in, about 74 in, or about 75 in. In some variations, the support unit 110 may comprise one or more of the same dimensions as a standard 19 U server rack, thereby enabling compatibility with standard rack-mountable components and accessories. In some variations, the support unit 110 may be dimensioned to accommodate custom instrument sizes, allowing for specialized processing instruments designed specifically for certain scales or types of nucleic acid manufacturing.

The support unit 110 may be configured to hold one or more instruments (e.g., processing instruments) in stacked or side-by-side orientations, thereby forming slots in which each instrument may be partially or entirely enclosed. In some variations, a plurality of slots may be arranged in one or more vertical columns (e.g., one column) within the housing. The support unit 110 may comprise about 4 to about 20 slots, such as about 6 to about 15, about 8 to about 13, or about 10 to about 11 slots (e.g., about 10 slots). A number of slots may be equal to a maximum number of instruments that the support unit 110 is configured to contain. Thus, the support unit 110 may be configured to house about 4 to about 20 instruments, such as about 6 to about 15, about 8 to about 13, or about 10 to about 11 instruments (e.g., about 10 instruments). The number of slots may be determined based on the overall dimensions of the support unit 110 and the height requirements of each instrument to be housed therein.

Each slot may be configured to contain an instrument (e.g., processing instrument) by contacting at least part of a perimeter, underside, and/or sidewall of the instrument. Each slot may additionally define a discrete volume within the housing. In some variations, a slot may comprise a support surface having a substantially planar configuration and being disposed substantially horizontally within the housing. For example, the support surface may comprise a base configured to directly support a base of an instrument thereon. In some variations, the support surface may comprise one or more retention elements (e.g., lips, flanges, or protrusions) extending therefrom to prevent movement of an instrument positioned thereon. These retention elements may be strategically positioned around the perimeter of the support surface to secure the instrument while minimizing interference with instrument operation or access.

Additionally, or alternatively, in some variations, a slot may comprise one or more arms (e.g., at least two arms, such as two opposing arms) mountable to a sidewall or rail of the housing and configured to support an instrument thereon. Each arm may comprise an angled lip that matches a contour of an instrument such that each arm may support the instrument along two sides thereof. The arms may be adjustable in terms of their vertical positioning within the housing, allowing the support unit to accommodate instruments of varying heights. The arms may also be removable or reconfigurable to adapt the support unit for different instrument types or arrangements.

In some variations, a slot may simply define the discrete volume of interior space of the housing without comprising additional structural elements. In such variations, the slot may not be a physical component but rather a designated portion of the interior space reserved for an instrument. For example, an instrument may be directly mountable against or coupled to the housing (e.g., a back sidewall and/or rail), whereby the instrument is secured within the support unit without requiring dedicated support surfaces or arms. The housing itself thus may provide the necessary structural support via direct coupling with the instrument. The slot, in this context, may refer to the three-dimensional volume that the instrument occupies when installed. In some variations, the volume of interior space may comprise a height that is about equal to or greater than a height of an instrument that the housing is configured to receive. In some variations, the height of the discrete volume may be about 2 U to about 9 U, such as about 3 U to about 8 U, about 4 U to about 6 U, about 2 U, about 3 U, about 4 U, about 5 U, about 6 U, about 7 U, or about 8 U. Put another way, each slot may comprise a height of about 4 in to about 10 in, such as about 5 in to about 9 in, about 6 in to about 8 in, or about 7 in. In some variations, each discrete volume may be greater than a height of a corresponding instrument such that a gap or clearance distance may be maintained between neighboring instruments. The clearance distance may be, for example, about 0.01 cm to about 10 cm (e.g., about 0.05 cm to about 5 cm, about 0.5 cm to about 2.5 cm, or about 1 cm to about 2 cm). In some variations, the housing may comprise one or more spacing elements configured to maintain clearance distances between adjacent instruments positioned within the support unit. The spacing elements may be integrally formed with the support unit structure or may be removably attached thereto. In some variations, the housing may not be configured to maintain the gap between processing instruments, thereby allowing opposing (e.g., top and bottom, respectively) surfaces on adjacent instruments to be in contact.

A perspective view of a plurality of processing instruments 620A/620B each mounted within a support unit 610A/610B is shown in FIG. 6. The support units 610A/610B may orient all of the processing instruments 620A/620B therein in a same direction such that an opening of each processing instrument is facing an external environment (with respect to the internal environment of the support units 610A/610B). This singular-configuration of the processing instruments 620A/620B within the support units 610A/610B may help to simplify instrument/cartridge loading operations and thus simplify the overall system configuration. This uniform orientation may additionally facilitate automated and/or operator access to the instruments, as well as utility connections across a plurality of instruments.

In some variations, each support unit may be dedicated to a specific processing instrument size or type. For instance, a first support unit may carry processing instruments suitable for cartridges having fluid capacities on the order of milliliters, while a second support unit may house larger processing instruments configured for liter-scale cartridges. This specialization may optimize the use of space and resources for particular scales of operation. Alternatively, all support units may be uniform, and the system may be reconfigured or populated with whichever processing instrument types are required by a given bioprocessing workflow. The specific arrangement of support units (whether in lines, grids, circular arcs, or other shapes) may be determined by facility constraints and throughput needs. Furthermore, each support unit and the associated processing instruments may be arranged such that the enclosure maintains a partially or fully sterile environment, helping safeguard each manufacturing batch. The support units may incorporate features such as sealed edges, gaskets, or air pressure differentials that contribute to maintaining the desired environmental conditions within the overall enclosure.

### Configuration

The system 100 may comprise a plurality of instruments and a plurality of support units that are arranged in a hierarchical configuration to optimize space within the system. At the primary (lowest) level of the hierarchy, each instrument may independently execute an automated workflow using a dedicated cartridge. This modularity allows for individual process control and isolation between manufacturing runs. At the secondary level, a plurality of instruments may be physically installed in a single support unit. This grouping provides efficient space utilization and shared infrastructure such as power distribution, data connectivity, or cooling systems. At the tertiary level, a plurality of support units may form a row, or “cluster.” These clusters may share common resources like robotic handling systems, operator access paths, or utility connections. At the quaternary (highest) level of the hierarchy, a plurality of clusters may be arranged in an array. This arrangement may provide for a scalable, high-throughput manufacturing environment that may be expanded or reconfigured as production demands change.

Referring to the tertiary level, a plurality of support units may be positioned side by side in a row, forming a cluster of processing capacity. In some configurations, a single row or cluster may include about 5 to about 15 support units, each supporting a plurality of processing instruments. For instance, if each structure houses about 10 processing instruments in total, and a row includes 10 support units, that cluster may comprise up to 100 processing instruments. Alternatively, larger clusters may comprise about 15 or more support units in a row, each structure holding 10 or more processing instruments, enabling parallel operation of over a hundred manufacturing workflows at once within the same area. The support units may be arranged so that adjacent sidewalls are in contact or nearly touching, maximizing floor-space efficiency and creating a continuous working plane along the length of the cluster. In other variations, small gaps or aisles may be left between support units for maintenance access, robotic arms, or cable routing. These gaps may be standardized across the facility to ensure consistent access for maintenance personnel or automated systems.

At the highest level, the system 100 may include a plurality of clusters of support units arranged in an array throughout an enclosure or facility. For example, an array may comprise 2 to about 5 rows (clusters). In some variations, the array may be organized in a rectangular grid, with two or more parallel rows of clusters separated by an aisle or corridor for robotic and/or operator access. The width of these aisles may be determined based on the specific requirements for material movement, operator workflow, or automated transport systems. Alternatively, the arrangement may be more complex, such as a U-shaped, L-shaped, or modular format where a plurality of arrays of clusters occupy different areas of the facility but remain under coordinated control. These non-linear arrangements may adapt to existing building layouts or optimize workflow between different processing stages. The resulting layout may allow tens, dozens, or even hundreds of processing instruments (each with its own cartridge-based workflow) to operate in parallel, all while sharing centralized resources such as a control system, a robotic transport system, or an environmental maintenance system (e.g., comprising an HVAC system). This centralization of shared resources may reduce overall system cost and complexity while maintaining individual process control for each cartridge-based manufacturing run.

Moreover, the modular and hierarchical nature of this system architecture may allow for dynamic reconfiguration, enabling the addition, removal, or repositioning of instruments and support units without disrupting ongoing operations. For instance, individual support units may be quickly integrated into an existing cluster or removed for maintenance, replacement, or upgrades, thereby minimizing downtime. Further, clusters and arrays may be flexibly arranged to adapt to existing facility layouts or to accommodate future expansion, operational scaling, or throughput adjustments. This scalability may support incremental growth from initial research-scale deployments through large-scale commercial production facilities, offering enhanced flexibility and adaptability across diverse therapeutic manufacturing applications.

FIG. 11 depicts a top view of a rendering of an exemplary arrangement of support units. As shown, a facility (e.g., the enclosure 101) may comprise an area of about 6 m by about 10 m. Still, 5 clusters—each comprising 10 support units, and each of those comprising 10 processing instruments—may fit in the area. This example demonstrates how the hierarchical organization enables high-density processing capacity within a relatively modest footprint, potentially supporting up to 500 parallel manufacturing operations in this configuration.

Because each cluster may be formed by aligning a plurality of support units, and because a plurality of clusters may be placed in an array without substantial redesign of individual racks or instruments, the facility may be scaled up or down rapidly to accommodate changing production demands. The modular nature of the system facilitates expansion without disrupting existing operations or requiring complete facility redesign. A relatively small site may use just one row (i.e., a single cluster) of three or four support units, each housing a handful of instruments, whereas a larger commercial facility may deploy a plurality of rows of at least five or at least ten (e.g., ten or more) support units, each structure configured to carry a corresponding number of instruments, for a total of hundreds of parallel manufacturing workflows. By leveraging the same base components (instruments, support units) repeated in a hierarchical fashion, the entire system remains standardized and straightforward to maintain, and the overall enclosure footprint may be optimized for GMP or other regulatory compliance standards. This standardization may extend to maintenance procedures, spare parts inventory, operator training, and validation protocols, potentially reducing operational complexity despite the high number of parallel processing units.

### VI. Control System

The system may comprise a control system that includes one or more controllers for monitoring, managing, and documenting all stages of the manufacturing process. In some variations, a master controller may coordinate multiple instruments and dynamically schedule tasks among them, while additional local controllers may direct real-time actuation within each instrument. This architecture may enable parallel processing of multiple patient-specific biotherapeutics while preserving traceability and preventing cross-contamination.

In some variations, the control system may comprise a hierarchical framework in which a manufacturing execution system (MES) orchestrates production activities (such as scheduling, load balancing, and resource allocation), while each instrument's local controller supervises on-board processes. The MES may retrieve patient-specific parameters from a higher-level database or prescription record, then relay them to the relevant instrument to configure workflow steps. The control system may thus unify the three-instrument workflow by ensuring that each stage receives the correct cartridge data, manufacturing parameters, and quality control requirements.

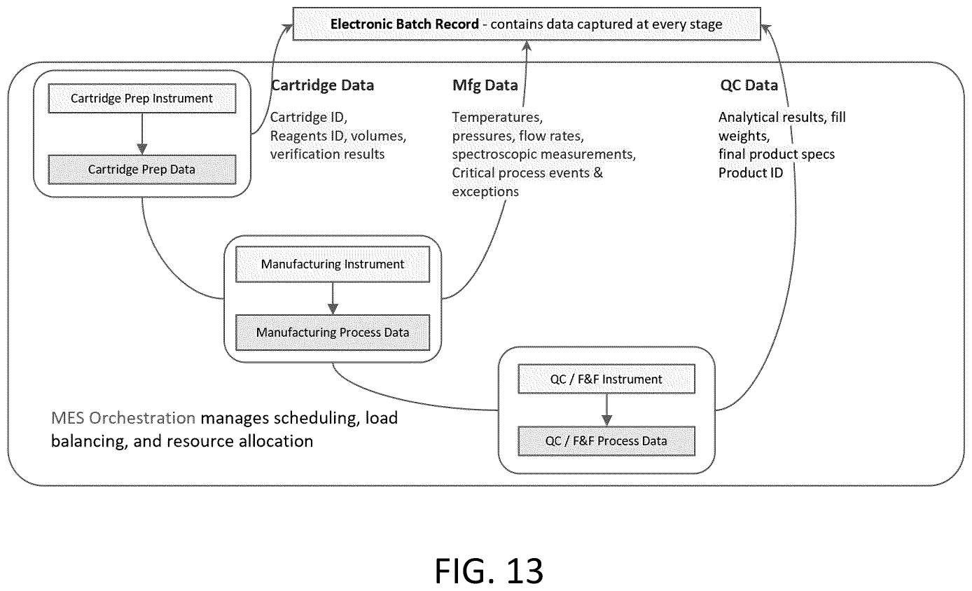

As shown in FIG. 13, the control system may integrate data from several categories into a unified electronic batch record (EBR). Cartridge data (e.g., reagent identification, fill volumes, expiration dates, and environment verifications) may be generated by a first (preparation) instrument and stored under a unique cartridge identifier. Manufacturing data (e.g., temperatures, pressures, flow rates, and spectroscopic measurements) may be collected from a second (processing) instrument that executes the core bioprocessing steps. QC and fill/finish data (e.g., analytical test results, fill weights, or container-closure specs) may be produced by a finishing instrument. The control system may continuously capture, validate, and merge all of this information, creating a comprehensive documentation package for each patient's batch. In some variations, the system may use 21 CFR Part 11—compliant software, ensuring that audit trails, electronic signatures, and access controls meet regulatory requirements. Additionally, data related to process deviations, alarms, or exceptions may be stored alongside normal process data, facilitating real-time corrective actions or final product disposition decisions.

In some variations, the control system may employ a conditional execution method, adjusting workflows based on sensor feedback from any stage of the manufacturing process. For example, if a temperature reading at a transcription step drifts outside specified limits, the control system may automatically pause, reconfigure the operating conditions, or alert an operator for manual intervention. By leveraging these feedback loops, the system may improve yield consistency and adapt to minor fluctuations without sacrificing product quality.

The control system 140 may further handle inventory management, reagent usage forecasting, and automated reordering of consumables. Such functionality may rely on data from each instrument, including the volumes dispensed, material lot numbers, and consumption rates. The system may store this information, along with environmental monitoring logs and maintenance records, to provide a full picture of operational efficiency and compliance. Each transaction or event may be linked to a particular batch record, preserving traceability and facilitating post-run analysis. The communication architecture (e.g., wired or wireless networks) may connect the control system to external databases, ensuring that prescription updates, newly identified targets, or revised protocols are propagated seamlessly throughout the facility.

Referring to FIG. 1F, the control system 140 (e.g., controller or computing device) may include one or more of a processor 142, memory 144, communication device, 146, input device 148, and display 150. A processor of the system controller (e.g., processor 142) may process data and/or other signals to control one or more components of the system. The processor 142 may be configured to receive, process, compile, compute, store, access, read, write, and/or transmit data and/or other signals. Additionally, or alternatively, the processor may be configured to control one or more components of a device (e.g., console, touchscreen, personal computer, laptop, tablet, server).

In some variations, the processor 142 may be configured to access or receive data and/or other signals from one or more of instruments, robots (e.g., of the RTS 104), servers, external control systems, and storage mediums (e.g., memory, flash drive, memory card, database). In some variations, the processor 142 may be any suitable processing device configured to run and/or execute a set of instructions or code and may include one or more data processors, image processors, graphics processing units (GPU), physics processing units, digital signal processors (DSP), analog signal processors, mixed-signal processors, machine learning processors, deep learning processors, finite state machines (FSM), compression processors (e.g., data compression to reduce data rate and/or memory requirements), encryption processors (e.g., for secure wireless data transfer), and/or central processing units (CPU). The processor 142 may be, for example, a general-purpose processor, Field Programmable Gate Array (FPGA), an Application Specific Integrated Circuit (ASIC), a processor board, and/or the like. The processor may be configured to run and/or execute application processes and/or other modules, processes and/or functions associated with the system. The underlying device technologies may be provided in a variety of component types (e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and the like.

The processor 142 may operate the systems/perform the methods herein using software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including structured text, typescript, C, C++, C#, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

The memory 144 may be configured to store data and/or information. In some variations, the memory may include one or more of a random-access memory (RAM), static RAM (SRAM), dynamic RAM (DRAM), a memory buffer, an erasable programmable read-only memory (EPROM), an electrically erasable read-only memory (EEPROM), a read-only memory (ROM), flash memory, volatile memory, non-volatile memory, combinations thereof, and the like. In some variations, the memory may store instructions to cause the processor to execute modules, processes, and/or functions associated with the device, such as image processing, image display, sensor data, data and/or signal transmission, data and/or signal reception, and/or communication. Some variations described herein may relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The computer code (also may be referred to as code or algorithm) may be those designed and constructed for the specific purpose or purposes. In some variations, the memory may be configured to store any received data and/or data generated by the controller. In some variations, the memory may be configured to store data temporarily or permanently.

The input device 148 may comprise or be coupled to a display (e.g., display 150). The input device 148 may be any suitable device that is capable of receiving input from an operator via, for example, a keyboard, buttons, touch screen, and/or the like. The input device may include at least one switch configured to generate a user input. For example, an input device may include a touch surface for a user to provide input (e.g., finger contact to the touch surface) corresponding to a user input. An input device including a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In variations of an input device including at least one switch, a switch may have, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive user movement data from an optical sensor and classify a user gesture as a user input. A microphone may receive audio data and recognize a user voice as a user input.

Graphical and/or image data may be output on the display 150 of the controller. In some variations, a display may include at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, and/or holographic display. In some variations, a GUI may be configured for designing a process and monitoring a product and may be shown on the display.

Further, the communication device 126 may be configured to communicate with another controller and one or more databases. The communication device may be configured to connect the controller to another system (e.g., Internet, remote server, database) by wired or wireless connection. In some variations, the system may be in communication with other devices via one or more wired and/or wireless networks. In some variations, the communication device may include a radiofrequency receiver, transmitter, and/or optical (e.g., infrared) receiver and transmitter configured to communicate with one or more devices and/or networks. The communication device may communicate by wires and/or wirelessly.

Alternative variations of the three-instrument system described above may include variations in the specific arrangement and configuration of the workflow components. In one alternative variation, the cartridge preparation and therapeutic manufacturing functions may be combined into a single instrument that performs both reagent loading and subsequent processing, followed by transfer to a separate finishing instrument. This configuration may be advantageous in smaller facilities with limited space requirements.

In another alternative variation, the quality control functions provided by the analytical system of the finishing instrument may be separated from the fill/finish operations of the filling system. This may provide a four-instrument workflow with additional parallelization capabilities for high-throughput facilities. The system may also be adapted to incorporate continuous manufacturing principles for certain process steps, such as implementing continuous chromatography for purification or continuous mixing for lipid nanoparticle formation.

The system may further be adapted to process alternative therapeutic modalities beyond mRNA, including other nucleic acid-based products such as siRNA, antisense oligonucleotides, or CRISPR components. With appropriate module modifications, the same architectural principles may extend to cell therapy manufacturing, protein therapeutics, or other advanced therapy medicinal products requiring personalized processing.

Overall, through these integrated approaches using specialized instruments, closed-system cartridges, and centralized control architecture, the system herein may provide a complete solution for manufacturing personalized nucleic acid therapeutics. The systems may enable parallel processing of multiple patient-specific products while maintaining sterility, traceability, and regulatory compliance throughout the entire workflow from initial reagent preparation to final drug product packaging. This modular, scalable approach may advance biomanufacturing technology and help make personalized mRNA therapeutics commercially viable at scales previously unattainable with traditional manufacturing methods.

### Methods for Automated Bioprocessing

The methods herein may generally relate to automated processes for producing nucleic acid-based therapeutics (e.g., mRNA products) and may be executed by any of the systems, instruments, and cartridges described herein. While these methods are illustrated with respect to certain steps arranged in a particular order, it should be understood that some steps may be modified, omitted, repeated, or rearranged without departing from the scope of the disclosure. Additional steps may also be included. In many variations, such methods may be used to produce patient-specific mRNA therapeutics and may be performed with minimal operator intervention, often within about 5 hours to about 15 hours of start time (e.g., within about 6 to about 14 hrs, about 7 to about 13 hrs, about 8 to about 12 hrs, about 9 to about 11 hrs, or about 10 hrs).

FIGS. 12A and 12B both illustrate an exemplary automated bioprocessing workflow 1200 for producing a nucleic acid product (e.g., an mRNA-based therapeutic) from a cartridge. As shown, the workflow 1200 may comprise a series of steps carried out by three classes of instruments—a cartridge preparation instrument, a processing instrument, and a final drug product QC and fill/finish instrument—and may conclude with a filled therapeutic product suitable for patient use and a spent cartridge for disposal.

At step 1201, a blank cartridge may be delivered into the system enclosure, for example via a feedthrough or loading station. The blank cartridge may be an unsealed, substantially empty unit that comprises structural features (e.g., reaction chambers, fluidic paths) but does not yet contain reagents. A robotic transfer system (RTS) or an operator may place this blank cartridge into the cartridge preparation instrument at step 1202 to begin the initial loading and sealing processes. The cartridge preparation instrument may confirm cartridge identity by scanning an RFID tag or barcode, then retrieve a patient-specific or batch-specific recipe from the control system. Bulk reagents (e.g., buffers, enzymes, and nucleotides) may be dispensed into designated chambers of the cartridge using precision fluid handling methods (such as micropipetting or low-flow pumping) while the instrument maintains an aseptic environment. After filling, the instrument may seal the cartridge by applying heat, pressure, or ultrasonic welding to close any fluid ports, thereby producing a filled or pre-processed cartridge. In some variations, the preparation instrument may also perform a degassing or conditioning routine—such as gently agitating certain reagents or adjusting temperatures—to ensure that each fluid compartment is fully primed for subsequent operations. The sealed, reagent-filled cartridge may then be checked for integrity (e.g., verifying no leaks or unsealed ports) before being transferred onward.

At step 1203, the filled, pre-processed cartridge may be conveyed (e.g., by the RTS) to a processing instrument for core nucleic acid operations. Upon arrival at this instrument, the cartridge may be aligned within a receiving bay, which may include mechanical connectors to hold the cartridge in place during processing. The instrument may confirm that the correct cartridge has been received (e.g., by reading its RFID tag), verify that reagents therein have not exceeded expiration, and validate any preconfigured workflow parameters. Because the cartridge may be sealed and preloaded, there may be no external fluid additions, lines, or sample ports required throughout manufacturing.

At step 1204, the processing instrument may carry out one or more nucleic acid processing steps in a closed, automated workflow. For example, the instrument may initiate thermal preconditioning, which rapidly brings specific fluid compartments of the cartridge to set temperatures using instrument-based thermal elements (e.g., Peltier modules or fluid-cooled plates). Once these compartments are at their designated setpoints (e.g., about 60-95° C. for amplification steps, about 30-50 ° C. for transcription, about 4-8° C. for reagent storage), the instrument may activate various actuating modules to execute each stage of processing. For example, during an amplification step, a DNA template may be amplified via polymerase chain reaction in a dedicated fluid compartment with high thermal conductivity walls. The temperature cycling profile may be precisely controlled, and sensors (e.g., UV or fluorescence) may provide real-time feedback on reaction progression. During a purification step, the amplified DNA may be transferred to a magnetic separation or tangential flow filtration submodule via a fluid transfer system (FTS). For example, the instrument may deliver controlled bursts of compressed air (or inert gas) into the cartridge to move fluid from one chamber to another, with valves and flow regulators ensuring accurate volumetric transfers. The purified DNA may be collected in a downstream chamber and readied for transcription. Subsequently, during a transcription step, the purified DNA, along with enzymes, nucleotides, and buffers, may be combined in a thermally controlled reaction chamber to generate mRNA. The instrument may maintain about 37° C. for optimal enzyme activity, continuously monitoring parameters such as pH and conductivity. Additionally, during a post-transcription treatment step (e.g., including digestion, capping & tailing, any unwanted template or partial sequences may be removed, and enzymatic capping/tailing steps may be performed as needed. The instrument may direct fluids through specific chambers while controlling temperature, mixing speeds, and reaction times. Further, during formulation and/or concentration steps, the intermediate mRNA product may be buffer-exchanged or concentrated (e.g., through tangential flow filtration), then combined with lipid solutions in a dedicated particle formation module to yield mRNA-loaded lipid nanoparticles (LNPs). Flow rates, cooling, and mixing conditions may be carefully regulated to achieve a target particle size.

Throughout these processing steps, sensors within the cartridge and/or processing instrument may provide real-time data (temperature, pressure, flow rate, UV absorbance, etc.) to the control system for closed-loop adjustments. For instance, if a pressure reading spikes during filtration, the control system may temporarily slow the gas flow or open a bypass valve to maintain consistent operating conditions. Moreover, all events and measurement logs may be automatically appended to an electronic batch record.

Upon completing the manufacturing steps, the processing instrument may store the final mRNA-lipid product in a cartridge compartment (e.g., a sealed reservoir) maintained at a suitable temperature (e.g., about 4° C.). The result of this stage may be a fully processed cartridge that contains the final nucleic acid product ready for fill/finish operations, with internal waste compartments containing any process byproducts. The control system 140 (or a local controller in the instrument) may notify the RTS or an operator that manufacturing is complete.

At step 1205, the fully processed cartridge may be transferred to a final drug product QC and fill/finish instrument for extraction, quality control, and packaging. The cartridge may once again be scanned upon insertion to verify identity and ensure that its corresponding manufacturing data are available. An aseptic connection may be established between the instrument and the sealed cartridge (e.g., by piercing a sterile port or docking a fluid transfer head). The finishing instrument may then extract the nucleic acid product into an intermediate container or directly into sample vessels for QC tests, such as measuring concentration, purity, or potency. Parallel to QC sampling, the instrument may dispense the remainder of the product into final containers (e.g., syringes, vials, or cryobags) under ISO 5/Grade A laminar airflow to maintain sterility. Container closure operations (e.g., crimping, capping, or stoppering) may also occur in this environment.

At step 1206, the finishing instrument may confirm that the final containers have been correctly sealed, optionally performing container closure integrity checks or optical inspections. In some variations, a controlled-rate freezing module may be engaged if the product requires cryopreservation at subzero temperatures. Temperature probes and logging devices may track the freezing profile for quality review. Meanwhile, the cartridge itself may become spent and may be considered waste or recyclable material once the product is fully extracted. A separate fluid line or vacuum system may remove residual wastes from the cartridge if needed, although in many variations the cartridge simply encloses its own waste compartments for safe disposal.

At step 1207, the filling and packaging are finalized, producing filled product in vials, syringes, or other containers suitable for patient administration. A labeling and packaging subsystem may attach patient-specific barcodes or RFID tags to each container, verifying label correctness (e.g., via a vision system). The finishing instrument or an external system may then compile the QC results, fill/finish data, and any additional environmental logs into the electronic batch record, ensuring traceability and regulatory compliance. The completed filled product may be cleared for shipment, and the spent cartridge may be removed from the instrument and routed for disposal. With these steps complete, the workflow 1200 ends, and the system may update all associated data repositories, including inventory management for reagent usage, temperature logs, and batch records tied to the patient's prescription.

Electronic batch records for a given cartridge may be generated at any point throughout pre-processing, processing, and post-processing. FIG. 13 depicts a representative data flow in which cartridge data from the first (preparation) instrument, manufacturing data from the second (processing) instrument, and QC/fill/finish data from the third (finishing) instrument may be converged into a unified EBR. First, the cartridge preparation instrument may generate “preparation data” comprising, for example, reagent volumes, verification of dispensing steps, and environment details (e.g., temperature or humidity logs). This cartridge data may be stored under a unique identifier. Second, as the cartridge enters the processing instrument, that identifier may link newly generated “manufacturing data” (e.g., reaction parameters, spectral measurements, flow rates, and intermediate yield confirmations) to the same record. Each sensor reading, event, and out-of-range condition may be timestamped and annotated.

Third, when the cartridge eventually moves to the finishing instrument, “QC and fill/finish data” may be added to the same record. Such data may encompass assay results (e.g., potency tests, purity assays, or residual DNA checks), fill-level verification, container-closure specifications, and final labeling details. A control system may orchestrate the EBR generation, and may, in some variations, note any in-process adjustments (such as an extended mixing time to optimize particle size) and record any exceptions or alarm events encountered. By merging all relevant data sources-cartridge, manufacturing, and QC/fill/finish-the EBR may form an unbroken chain of information for each patient-specific product.

In some variations, the control system may push real-time data to a higher-level MES, which orchestrates scheduling, load balancing, and resource allocation across multiple instruments or racks. The MES may further integrate these data streams into production scheduling software and LIMS, allowing automated triggers or flags if a parameter exceeds defined tolerances. In the event of critical deviations, the method may include an operator review step prior to re-engaging the workflow, ensuring that product integrity remains uncompromised.

Once the final mRNA product is encapsulated in LNPs and stored in a designated compartment of the cartridge, the processing method may conclude. Then, a finalized EBR may be generated. This final record may consolidate all sensor readings, user interventions, environmental logs, and process details into a single digital package. In some variations, the package may be 21 CFR Part 11—compliant. In some variations, the EBR may contain references to any calibration certificates, maintenance activities, or prior stage verifications performed on the cartridge. Because the EBR may aggregate data from distinct instruments, it may eliminate manual transcription errors and accelerate review processes.

In some variations, the finishing instrument may also add additional data for shipping or distribution, such as package temperature logs, container closures, or final product labeling. The control system 140 may append this information to the batch record, thereby forming a complete production history from cartridge preparation through final product release. The method may end with the EBR archived, validated, and optionally transmitted to a secure database or remote server for retention. Quality assurance personnel may then perform a final audit, electronically sign the record, and release the product for patient administration.

Throughout this flow, each instrument may contribute real-time data to the batch record as illustrated in FIG. 13. For instance, the preparation data block may feed forward into the manufacturing data which subsequently feeds into the QC and fill/finish data—all under a unified, orchestrated approach. The control system may apply built-in analytics or data reduction algorithms to identify subtle trends, exceptions, or patterns that could inform continuous improvement efforts. By embedding these robust data flows into an EBR, the methods described herein may fulfill regulatory requirements for patient-specific mRNA therapeutics while offering traceability and consistency far beyond that of manual, multi-stage systems.

Other methods enabled by the system herein may comprise product manufacturing—specific and/or parallel processing methods for automated bioprocessing. For example, in some variations, one such exemplary method may first include transferring reagents to a plurality of cartridges, each cartridge containing nucleic acid material. The reagents transferred may be specific to an intended workflow (e.g., in vitro transcription, purification, particle formation). Such transferring may occur in a sterile environment, and the cartridges may be sealed to maintain a closed environment for subsequent operations. Each cartridge may be engaged with a dedicated processing instrument in a set of processing instruments arranged for parallel processing. The engagement may involve positioning the cartridge in a receiving bay of the processing instrument so that one or more actuating modules of the processing instrument interface with the cartridge's internal modules. In certain instances, at least two cartridges may be engaged with two corresponding processing instruments, enabling simultaneous or otherwise parallel execution of distinct nucleic acid processing workflows. In some variations, the method may be performed at scale, for instance involving dozens or hundreds of instruments (each with a corresponding cartridge), all orchestrated by a control system that schedules reagent loading, monitors reagent inventories, and ensures that each cartridge receives its patient-specific target sequence or batch recipe.

Once engaged, each processing instrument may operate under a control system that maintains the closed environment of its corresponding cartridge throughout the workflow. The method may then include executing, in parallel, one or more processing steps on each nucleic acid material. These steps may entail transcription to produce mRNA from a DNA template, purification of the mRNA (such as through tangential flow filtration, magnetic bead separation, chromatography, or other means), and encapsulation of the mRNA in lipid nanoparticles. In various examples, the nucleic acid materials in different cartridges may have different target sequences, permitting a plurality of patient-specific therapeutics to be produced concurrently. In certain variations, these workflows may be executed without removing any cartridge from its processing instrument until the processing is complete. Each processing instrument may monitor conditions within its cartridge using one or more sensors that detect parameters such as temperature, pressure, and reagent flow. Such monitoring may occur continuously or intermittently to confirm that each workflow remains on track and that each final product meets one or more quality standards. Furthermore, if any sensor data indicate deviations from acceptable process thresholds (e.g., an unexpected pressure spike or a temperature drift), the control system may pause or alter the step in progress, log the event, and optionally alert operators to perform an intervention or override command.

The method may include producing a plurality of therapeutic products in parallel, each derived from distinct nucleic acid materials, including patient-specific target sequences. The total processing time for each product may range from about 5 hours to about 15 hours, depending on the specific protocol, the volume being processed, and other workflow parameters. In some variations, these methods may be extended to any suitable number of processing instruments operating simultaneously, thereby scaling up total throughput. Following completion, the method may include collecting or withdrawing each sealed cartridge from its processing instrument (e.g., via robotic or manual retrieval) and recovering each product for delivery to the appropriate patient or downstream processing entity. Throughout the procedure, a controller or network of controllers may record operational data and product parameters, facilitating creation of an electronic batch record or equivalent documentation for regulatory compliance. In some variations, this record may capture data at sub-minute intervals (or more frequently), covering all critical parameters such as reagent lot usage, sensor readings, and the timing of each workflow step.

Such methods may be performed in a wide range of facilities, including cleanrooms or other partially or fully sterile environments. Additionally, the entire workflow may be repeated or varied to accommodate different nucleic acid constructs, different target sequences, or different process end points (such as formulation into bulk product or direct fill operations). Each step may be automated to a desired degree, including reagent loading, cartridge insertion, workflow initiation, in-process sensing, and product removal, thereby providing a robust, closed, and parallelized approach to producing mRNA-based or other nucleic acid-based therapeutics. In some variations, a manufacturing execution system (MES) may oversee scheduling for multiple runs, handle inventory allocations, and automatically generate production metrics (e.g., yield, throughput) for each completed workflow. This holistic approach may allow the facility to adapt rapidly to changing patient demands, switch between diverse protocols, and track each product seamlessly throughout its lifecycle.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “about” are used as equivalents.

Additionally, it should be appreciated that ranges disclosed herein may be exemplary, and include all ranges and subranges therein.

While certain variations are described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive variations described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive variations described herein. It is, therefore, to be understood that the foregoing variations are presented by way of example only and that, within the scope of the appended claims and equivalents thereto; inventive variations may be practiced otherwise than as specifically described and claimed. Inventive variations of the present disclosure are directed to each individual feature and/or method described herein. In addition, any combination of two or more such features and/or methods, if such features and/or methods are not mutually inconsistent, is included within the inventive scope of the present disclosure.

1. A system for processing nucleic acid material, comprising:
a plurality of processing instruments, each processing instrument comprising a receiving bay and a plurality of actuating modules, wherein each processing instrument is configured to:
receive, in its receiving bay, a cartridge containing nucleic acid material; and
execute, through the plurality of actuating modules, a complete processing workflow on the nucleic acid material to produce a final nucleic acid product while the cartridge remains within the receiving bay, wherein the nucleic acid material is not transferred out of the cartridge or to another instrument during the complete processing workflow; and
a support unit that supports the plurality of processing instruments in a fixed arrangement,
wherein the system is configured to coordinate simultaneous processing of final nucleic acid products across the plurality of processing instruments within the support unit.

2. The system of claim 1 further comprising an enclosure housing the support unit and the plurality of processing instruments, wherein the enclosure is configured to maintain a sterile environment.

3. The system of claim 2, wherein the enclosure comprises one or more rooms.

4. The system of claim 1, wherein the one or more actuating modules comprise at least one of an amplification module, a transcription actuating module, a digestion actuating module, a capping & tailing actuating module, and a purification actuating module.

5. The system of claim 1, wherein the processing instrument further comprises a sensor system for monitoring the processing workflow.

6. The system of claim 5, wherein the sensor system comprises at least one of a temperature sensor, a camera, a conductivity sensor, a pH sensor, a pressure sensor, a flow sensor, an optical sensor, a spectroscopic sensor, a particle concentration sensor, and a particle size sensor.

7. The system of claim 1, wherein the processing instrument further comprises a first thermal element adjacent a first side of the receiving bay and a second thermal element adjacent a second, opposite side of the receiving bay.

8. The system of claim 1, wherein the receiving bay comprises at least one of a mechanical connector, an electrical connector, and a fluid connector configured to engage the cartridge.

9. The system of claim 1, wherein the processing instrument further comprises a vent within a sidewall thereof.

10. The system of claim 1, wherein the processing instrument further comprises a door coupled to an opening of the receiving bay.

11. The system of claim 10, wherein the door comprises a closed configuration and an open configuration, and wherein the door is in the closed configuration when the cartridge is within the processing instrument.

12. The system of claim 1, wherein the support unit comprises a frame or a rack.

13. The system of claim 1, wherein the support unit comprises a plurality of stacked slots.

14. The system of claim 13, wherein the slots are stacked in vertical columns.

15. The system of claim 13, wherein each slot comprises a height of about 5 in to about 10 in.

16. The system of claim 1, wherein the support unit comprises a height of about 15 in to about 30 in.

17. The system of claim 1, wherein the fixed arrangement comprises an array.

18. The system of claim 1 further comprising a plurality of support units.

19. The system of claim 18, wherein the plurality of support units is arranged in a cluster.

20. The system of claim 19, wherein the cluster comprises a row.

21. The system of claim 19 further comprising a plurality of clusters of support units.

22. The system of claim 1 further comprising a robot configured to transfer the cartridge to and from the processing instrument.

23. The system of claim 1 further comprising a controller communicably coupled to each of the plurality of actuating modules.

24. The system of claim 23, wherein the controller is configured to generate an electronic batch record for the final nucleic acid product of the system.

25. The system of claim 1, wherein the nucleic acid material comprises material to form mRNA.

26.-60. (canceled)

61. The system of claim 1, wherein the cartridge is sealed and maintains a closed internal environment throughout the complete processing workflow.

62. The system of claim 1, wherein the complete processing workflow comprises at least amplification, transcription, purification, and formulation of the final nucleic acid product.
